(19) United States
(12) Patent Application Publication
(10) Pub. No.: US 2022/0140332 A1
(43) Pub. Date: May 5, 2022

(54) ANALYSIS METHOD OF LITHIUM COMPOSITE OXIDE, POSITIVE ELECTRODE ACTIVE MATERIAL, AND SECONDARY BATTERY

(71) Applicant: SEMICONDUCTOR ENERGY LABORATORY CO., LTD., Atsugi-shi, Kanagawa-ken (JP)

(72) Inventors: Tatsuyoshi TAKAHASHI, Atsugi, Kanagawa (JP); Mayumi MIKAMI, Atsugi, Kanagawa (JP); Yohei MOMMA, Isehara, Kanagawa (JP); Jo SAITO, Atsugi, Kanagawa (JP)

(21) Appl. No.: 17/423,138

(22) PCT Filed: Jan. 22, 2020
(86) PCT No.: PCT/IB2020/050459

§ 371 Date: Jul. 15, 2021

(30) Foreign Application Priority Data
Jan. 31, 2019 (JP) 2019-015036

(51) Int. Cl. H01M 4/525

(52) U.S. Cl. CPC H01M 4/525

(57) ABSTRACT

An analysis method of a lithium composite oxide is provided. The method is to analyze substitution positions of a Ni atom and a Mg atom in a compound represented by a chemical formula Li(1−x−y)Co(1−a−b)Ni(x+a)Mg(y+b)O2. The analysis method includes a first calculation step of calculating stabilization energy when a Ni atom and a Mg atom each substitute for a Li atom and/or a Co atom contained in a LiCoO2 crystal. The analysis method includes a second calculation step of calculating stabilization energy of the compound represented by the chemical formula when cation occupancy of Li sites is changed. The analysis method includes a first measurement step of measuring charge-discharge efficiency in the first cycle and charge-discharge efficiency in the n-th cycle of the compound represented by the chemical formula. Note that n means an integer greater than or equal to 2.

### TECHNICAL FIELD

One embodiment of the present invention relates to a method of analyzing a lithium composite oxide. One embodiment of the present invention relates to an object or a manufacturing method. One embodiment of the present invention relates to a process, a machine, manufacture, or a composition of matter. One embodiment of the present invention relates to a semiconductor device, a display device, a light-emitting device, a power storage device, a lighting device, an electronic device, or a manufacturing method thereof.

Note that in this specification, a power storage device refers to every element and device having a function of storing power. Examples of the power storage device include a storage battery (also referred to as a secondary battery) such as a lithium-ion secondary battery, a lithium-ion capacitor, and an electric double layer capacitor.

In addition, electronic devices in this specification mean all devices including power storage devices, and electro-optical devices including power storage devices, information terminal devices including power storage devices, and the like are all electronic devices.

### BACKGROUND ART

In recent years, a variety of power storage devices such as lithium-ion secondary batteries, lithium-ion capacitors, and air batteries have been actively developed. In particular, demands for lithium-ion secondary batteries with high output and high capacity have rapidly grown with the development of the semiconductor industry, for portable information terminals such as mobile phones, smartphones, and laptop computers; portable music players; digital cameras; medical equipment; next-generation clean energy vehicles such as hybrid electric vehicles (HEV), electric vehicles (EV), and plug-in hybrid electric vehicles (PHEV); and the like. The lithium-ion secondary batteries are essential as rechargeable energy supply sources for the modern information society.

Thus, improvement of a positive electrode active material has been studied to increase the cycle characteristics and the capacity of the lithium-ion secondary battery (Patent Document 1 and Patent Document 2).

The performance required for power storage devices includes safe operation under a variety of environments and longer-term reliability.

### REFERENCE

### Patent Document

[Patent Document 1] Japanese Published Patent Application No. 2012-018914
[Patent Document 2] Japanese Published Patent Application No. 2016-076454

### Non-Patent Document

[Non-Patent Document 1] Toyoki Okumura et al., “Correlation of lithium ion distribution and X-ray absorption near-edge structure in O3- and O2-lithium cobalt oxides from first-principle calculation”, Journal of Materials Chemistry, 2012, 22, pp. 17340-17348.
[Non-Patent Document 2] Motohashi, T. et al., “Electronic phase diagram of the layered cobalt oxide system LiₓCoO₂ (0.0≤x≤1.0)”, Physical Review B, 80 (16), 2009, 165114.

### SUMMARY OF THE INVENTION

### Problems to be Solved by the Invention

Various aspects of lithium ion secondary batteries and positive electrode active materials used in them such as capacity, cycle performances, charge-discharge characteristics, reliability, safety, and cost are desired to be improved; a lithium composite oxide LiMO₂ (M is two or more metals including Co), in which a part of LiCoO₂ is substituted by other elements, has been developed.

However, the number of the substitution elements is very small; the method to reveal the structure of the lithium composite oxide has not been invented yet. If the structure of the lithium composite oxide is revealed, it may help to develop materials and to clarify the mechanisms such as charge-discharge characteristics and reliability.

In view of the above, an object of one embodiment of the present invention is to provide a method to analyze a lithium composite oxide. An object is to provide a positive electrode active material particle which hardly deteriorates. An object of one embodiment of the present invention is to provide a novel positive electrode active material particle. An object of one embodiment of the present invention is to provide a power storage device which hardly deteriorates. An object of one embodiment of the present invention is to provide a highly safe power storage device. An object of one embodiment of the present invention is to provide a novel power storage device.

Note that the description of these objects does not preclude the existence of other objects. One embodiment of the present invention does not have to achieve all these objects. Note that other objects can be taken from the description of the specification, the drawings, and the claims.

### Means for Solving the Problems

One embodiment of the present invention is a method to analyze substitution positions of a Ni atom and a Mg atom in a compound represented by a chemical formula Li_{(1-x-y)}Co_{(1-a-b)}Ni₍ₓ₊ₐ₎Mg_{(y+b)}O₂. The method includes a first calculation step of calculating stabilization energy of the compound represented by the chemical formula when a Ni atom and a Mg atom each independently substitute for a Li atom and/or a Co atom contained in a LiCoO₂ crystal. The method includes a second calculation step of calculating the stabilization energy of the compound represented by the chemical formula when cation occupancy of Li sites is changed. The method includes a first measurement step of measuring charge-discharge efficiency in the first cycle and charge-discharge efficiency in the n-th cycle of the compound represented by the chemical formula (n is an integer greater than or equal to 2). In the chemical formula, x+y<1, a+b<1, and x, y, a, and b each independently represent a real number greater than or equal to 0 and less than or equal to 1.

In the above structure, the measurement step includes at least a step of making a sample and a step of mechanical measurement. Owing to a combination of calculation and measurement, calculation results and the validity of events expected from the calculation results can be evaluated, which enables detail analysis.

In the above structure, in the first calculation step and the second calculation step, a GGA+U(DFT-D2) method is preferably used.

In the above structure, the cation occupancy is preferably changed at least within a range of 80% to 100% for calculation

In the above structure, n is preferably 2.

In the above structure, it is preferable that in the chemical formula, 0<x+a≤0.015 and 0<y+b≤0.06

In the above structure, in the second calculation step, when the Ni atom and the Mg atom substitute for the same kind of atoms in the LiCoO₂ crystal, the stabilization energy of the case where the same kind of atoms exists in the same layer in the LiCoO₂ crystal and the stabilization energy of the case where the same kind of atoms exists in different layers in the LiCoO₂ crystal are preferably calculated.

### Effect of the Invention

According to one embodiment of the present invention, a method to analyze a lithium composite oxide can be provided. A positive electrode active material particle which hardly deteriorates can be provided. A novel positive electrode active material particle can be provided. A power storage device which hardly deteriorates can be provided. A highly safe power storage device can be provided. A novel power storage device can be provided.

### BRIEF DESCRIPTION OF THE DRAWINGS

FIG. 1 is a diagram showing an example of a crystal structure of LiCoO₂.

FIG. 2A and FIG. 2B are diagrams showing examples of crystal structures of a lithium composite oxide.

FIG. 3 is a diagram showing an example of a manufacturing method of a positive electrode active material.

FIG. 4 is a diagram showing crystal structures and magnetism of a positive electrode active material of a conventional example.

FIG. 5 is a diagram showing crystal structures and magnetism of a positive electrode active material.

FIG. 6A and FIG. 6B are cross-sectional views of an active material layer containing a graphene compound as a conductive additive.

FIG. 7A and FIG. 7B are diagrams showing a coin secondary battery.

FIG. 8A and FIG. 8B are diagrams showing cylindrical secondary batteries. FIG. 8C is a perspective view of a battery module. FIG. 8D is a top view of a battery module.

FIG. 9A and FIG. 9B are diagrams showing examples of a secondary battery.

FIG. 10A1, FIG. 10A2, FIG. 10B1, and FIG. 10B2 are diagrams showing examples of a secondary battery.

FIG. 11A and FIG. 11B are diagrams showing examples of a secondary battery.

FIG. 12A and FIG. 12B are diagrams showing examples of a secondary battery.

FIG. 13 is a diagram showing an example of a secondary battery.

FIG. 14A, FIG. 14B, and FIG. 14C are diagrams showing a laminated secondary battery.

FIG. 15A and FIG. 15B are diagrams showing a laminated secondary battery.

FIG. 16 is an external view of a secondary battery.

FIG. 17 is an external view of a secondary battery.

FIG. 18A, FIG. 18B, and FIG. 18C are diagrams showing a manufacturing method of a secondary battery.

FIG. 19A is a top view of a bendable secondary battery. FIG. 19B1 is a cross-sectional view along the broken line C1-C2. FIG. 19B2 is a cross-sectional view along the broken line C3-C4.

FIG. 19C is a cross-sectional view along the broken line A1-A2. FIG. 19D is a cross-sectional view along the broken line B1-B2 when the bendable battery is bent.

FIG. 20A is a perspective view of a secondary battery. FIG. 20B is a perspective view of a secondary battery.

FIG. 21A is a perspective view of an electronic device. FIG. 21B is a perspective view of the electronic device. FIG. 21C is a perspective view of a secondary battery. FIG. 21D is a perspective view of an electronic device. FIG. 21E is a perspective view of a secondary battery.

FIG. 21F and FIG. 21G are perspective views showing examples of electronic devices.

FIG. 22A is a top view of an open electronic device. FIG. 22B is a top view of a closed electronic device. FIG. 22C is a diagram showing a block diagram of an electronic device.

FIG. 23 is a diagram showing examples of electronic devices.

FIG. 24A, FIG. 24B, and FIG. 24C are diagrams showing examples of electronic devices.

FIG. 25 is a diagram showing the calculation results of the stabilization energy of Example.

FIG. 26 is a diagram showing the calculation results of the stabilization energy of Example.

FIG. 27 is a diagram showing the charge-discharge efficiency of Example.

### MODE FOR CARRYING OUT THE INVENTION

Embodiments of the present invention are described in detail below with reference to the drawings. Note that the present invention is not limited to the following description, and it is readily understood by those skilled in the art that modes and details of the present invention can be modified in various ways. In addition, the present invention should not be construed as being limited to the description of embodiments below.

In the crystallography, a bar is placed over a number in the expression of crystal planes and orientations; however, in this specification and the like, crystal planes and orientations are expressed by placing a minus sign (−) before a number because of expression limitations. Furthermore, an individual direction which shows an orientation in a crystal is denoted by “[ ]”, a set direction which shows all of the equivalent orientations is denoted by “< >”, an individual plane which shows a crystal plane is denoted by “( )”, and a set plane having equivalent symmetry is denoted by “{ }”.

### Embodiment 1

As a positive electrode material, a lithium composite oxide in which a part of LiCoO₂ is substituted by other elements has been developed. The number of the substitution elements is too small; it is difficult to analyze the crystal structure of the lithium composite oxide. The present inventors have found that the crystal structure can be analyzed efficiently and in detail through analysis of the crystal structure using a combination of chemical calculations and experiments.

### [Calculation of Stabilization Energy Different Between Substitution Positions]

When LiCoO₂ is doped with a metal element, a Li site or a Co site in a crystal of LiCoO₂ may be substituted by the metal element; however, at the moment, it is difficult to analyze which site is substituted through a measurement. In the case of doping with a plurality of metal elements, each doped metal may be substituted for a Li site or a Co site, which makes the situation complex. Thus, it is effective to estimate, through chemical calculation, the site for which the doped metal is substituted by calculating the stabilization energy of the crystal structure with an arrangement of cation sites. An analysis method of a substitution position of a doped element in a lithium composite oxide is described below using an example of a calculation on a material represented by the chemical formula Li_{(1-x-y)}Co_{(1-a-b)}Ni₍ₓ₊ₐ₎Mg_{(y+b)}O₂, which is an example of a lithium composite oxide in which LiCoO₂ is doped with Mg and Ni. In the above chemical formula, x+y<1 and a+b<1.

A concept of a crystal model is preferably made by an experimenter and calculations are preferably carried out with a computer. When a concept of a crystal model is made by an experimenter, relations between a crystal condition and parameters become clear, which enables the following analyses to be performed in detail. The calculation amount is enormous; calculation results can be obtained fast with a computer. The following calculation example is explained on the assumption that the crystal structure of LiCoO₂ (R-3m (O3)) is made by an experimenter and calculations are carried out with a computer.

[Calculation Example of Stabilization Energy of Li_{(1-x-y)}Co_{(1-a-b)}Ni₍ₓ₊ₐ₎Mg_{(y+e)}O₂]

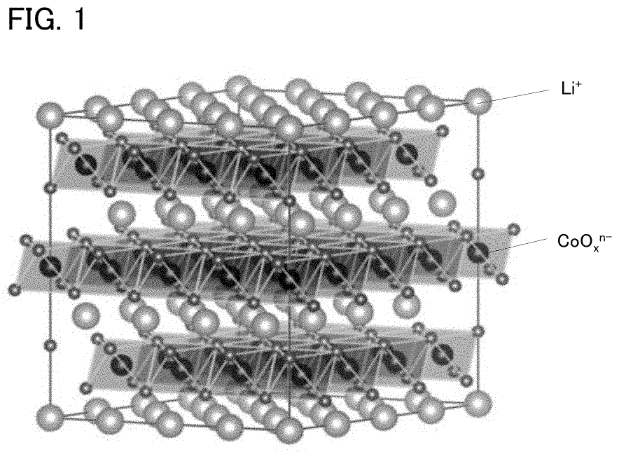

A model is made in which LiCoO₂ with a crystal structure of R-3m (O3), which is a basic structure of LiCoO₂, is doped with Mg and Ni, and the stabilization energy thereof is calculated; through this, the crystal structure of Li_{(1-x-y)}Co_{(1-a-b)}Ni₍ₓ₊ₐ₎Mg_{(y+b)}O₂ can be analyzed. FIG. 1 shows the crystal model of R-3m (O3), which is a basic structure of LiCoO₂. FIG. 1 shows the crystal structure of LiCoO₂ which consists of 48 Li atoms, 48 Co atoms, and 96 oxygen atoms (192 atoms in total).

Li_{(1-x-y)}Co_{(1-a-b)}Ni₍ₓ₊ₐ₎Mg_{(y+b)}O₂ is a compound in which Mg and Ni are used as substitution elements for (a) metal element(s) of LiCoO₂ (one or both of Li and metal). The following crystal models are considered as the combinations of the substitution sites of Mg and Ni.

Li in the same Li layer are substituted by Mg and Ni.
Li in different Li layers are substituted by Mg and Ni.
Co in the same Co layer are substituted by Mg and Ni.
Co in different Co layers are substituted by Mg and Ni.
Li in a Li layer is substituted by Mg and Co in a Co layer is substituted by Ni.
Li in a Li layer is substituted by Ni and Co in a Co layer is substituted by Mg.

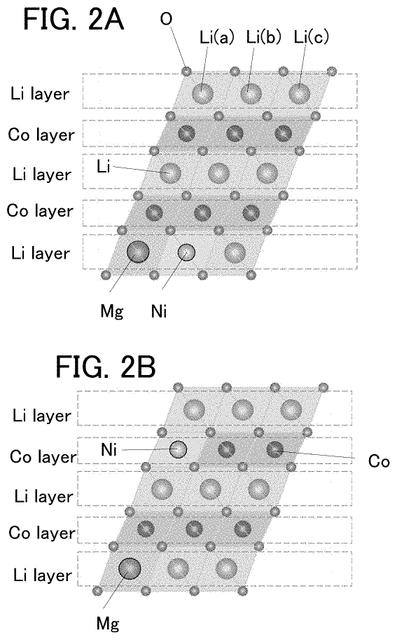

FIG. 2A and FIG. 2B show examples of substitution positions of Mg and Ni in a LiCoO₂ crystal. FIG. 2A shows that Li in the same Li layer are substituted by Mg and Ni and FIG. 2B shows that Li in different Li layers are substituted by Mg and Ni.

The stabilization energy of the crystal structure in each of the above combinations of the substitution sites is calculated, whereby what crystal structure Li_{(1-x-y)}Co_{(1-a-b)}Ni₍ₓ₊ₐ₎Mg_{(y+b)}O₂ can form can be analyzed. The stabilization energy can be estimated with the following formulae. A model is assumed here in which a Li atom and a Co atom are substituted by one Mg atom and one Ni atom in LiCoO₂ which consists of 48 Li atoms, 48 Co atoms, and 96 oxygen atoms (192 atoms in total).

<Case where Two Li Sites are Substituted by One Mg and One Ni>

(Equation 1)

ΔE=[E_total{(Li₄₆Mg₁Ni₁Co₄₈O₉₆)+2×E_atom(Li)}−{E_total(Li₄₈Co₄₈O₉₆)+E_atom(Mg)+E_atom(Ni)} (Formula 1)

<Case where One Li Site is Substituted by One Mg and Co Site is Substituted by One Ni>

(Equation 2)

ΔE=[E_total{(Li₄₇Mg₁Ni₁Co₄₇O₉₆)+E_atom(Li)+E_atom(Co)}−{E_total(Li₄₈Co₄₈O₉₆)+E_atom(Mg)+E_atom(Ni)} (Formula 2)

<Case where Two Co Sites are Substituted by One Mg and One Ni>

(Equation 3)

ΔE=[E_total{(Li₄₈Mg₁Ni₁Co₄₆O₉₆)+2×E_atom(Co)}−{E_total(Li₄₈Co₄₈O₉₆)+E_atom(Mg)+E_atom(Ni)} (Formula 3)

Symbols in Formula 1 to Formula 3 are as follows.

ΔE: stabilization energy.
E_total (Li₄₆Mg₁Ni₁Co₄₈O₉₆): in a LiCoO₂ crystal (192 atoms in total), the energy of the model in which two Li are substituted by one Mg and one Ni.
E_total (Li₄₇Mg₁Ni₁Co₄₇O₉₆): in a LiCoO₂ crystal (192 atoms in total), the energy of the model in which one Li is substituted by one Mg and one Co is substituted by one Ni.
E_total (Li₄₈Mg₁Ni₁Co₄₆O₉₆): in a LiCoO₂ crystal (192 atoms in total), the energy of the model in which two Co are substituted by one Mg and one Ni.
E_total (Li₄₈Co₄₈O₉₆): the energy of the model of a LiCoO₂ crystal (192 atoms in total).
E_atom (Li): the energy of one Li atom.
E_atom (Co): the energy of one Co atom.
E_atom (Mg): the energy of one Mg atom.
E_atom (Ni): the energy of one Ni atom.

The stabilization energy of each combination of the substitution sites by Mg and Ni is calculated and the results are compared, whereby a stable crystal structure can be estimated. The smaller the stabilization energy which is calculated from Formulae (1) to (3) is, the more stable the crystal model is and the more likely Li_{(1-x-y)}Co_{(1-a-b)}Ni₍ₓ₊ₐ₎Mg_{(y+b)}O₂ is to form the model.

The LDA+U method, the GGA+U(DFT-D2) method, or the like can be used for the calculation; the GGA+U(DFT-D2) method is preferably used. The GGA+U(DFT-D2) method can accurately estimate the stabilization energy since the GGA+U(DFT-D2) method can more accurately estimate the Van der Waals force than the LDA+U method.

Stabilization energy is influenced by the position where an atom is substituted. For example, in FIG. 2A, stabilization energy may differ between the situation in which Mg is substituted for the Li(a) site and Ni is substituted for the Li(b) site (first proximity) and the situation in which Mg is substituted for the Li(a) site and Ni is substituted for the Li(c) site (second proximity) though Li in the same layer are substituted by Mg and Ni. To calculate the stabilization energy, the positional relation between substitution atoms as well as the layer in which substituted atoms existed is preferably considered. When which layer Mg or Ni substitutes for is analyzed, in the six crystal models shown above, the stabilization energy of the first proximity to that of the third proximity of the crystal models to be compared are calculated and are compared, which is preferable. It is more preferable to calculate the stabilization energy of the first proximity to that of the fourth proximity and compare the results.

In the above formula, a model having 192 atoms in a crystal is used for the calculation; a user of this invention can extend the tendency of the calculation result to the bulk crystal. It is assumed that a model with a different number of atoms shows a similar tendency; the tendency of the calculation result can also be extended to a model with a different number of atoms. For example, when a model in which Li in the same Li layer are substituted by Mg and Ni has a lower stabilization energy than a model in which Co in the same Co layer are substituted by Mg and Ni, a similar tendency is assumed to be shown in the bulk crystal and a model with a different number of atoms; the tendency of the calculation result can be extended.

The stabilization energy of each crystal model is calculated and the results are compared as described above; through this, what crystal structure Li_{(1-x-y)}Co_{(1-a-b)}Ni₍ₓ₊ₐ₎Mg_{(y+b)}O₂ forms can be analyzed. The calculation and the comparison of the stabilization energy estimate a lithium composite oxide with a cation site occupancy of 100%; however, behavior and change in the crystal structure of the lithium composite oxide used to a positive electrode are not considered on calculation. When the lithium composite oxide is used as a positive electrode material, behavior during charging and discharging is preferably considered. Considering behavior during charging and discharging enables detailed analysis to be conducted.

[Calculation of Stabilization Energy when Cation Occupancy of Li Site is Changed]

In order to estimate a change of the crystal structure of a lithium composite oxide during charging and discharging a secondary battery when the lithium composite oxide is used as a positive electrode material of the secondary battery, the stabilization energy of the lithium composite oxide from which Li is extracted, that is, the stabilization energy when cation occupancy of Li sites is changed is calculated. Extraction of Li from a lithium composite oxide is assumed to be behavior occurring in a positive electrode of a secondary battery during charging. The stabilization energy of a lithium composite oxide with Li extracted is estimated as the stabilization energy during charging.

Formulae to calculate the stabilization energy when cation occupancy of Li sites is changed are shown below.

<Case where Two Li Sites are Substituted by One Mg and One Ni>

(Equation 4)

ΔE_{C}=[E_{C}_total{(Li_{46-z}Mg₁Ni₁Co₄₈O₉₆)+z×E_atom(Li)}−{E_total(Li₄₈Co₄₈O₉₆)+E_atom(Mg)+E_atom(Ni)} (Formula 4)

<Case where One Li Site is Substituted by One Mg and Co Site is Substituted by One Ni>

(Equation 5)

ΔE_{C}=[E_{C}_total{(Li_{47-z}Mg₁Ni₁Co₄₇O₉₆)+z×E_atom(Li)+E_atom(Co)}−{E_total(Li₄₈Co₄₈O₉₆)+E_atom(Mg)+E_atom(Ni)} (Formula 5)

<Case where Two Co Sites are Substituted by One Mg and One Ni>

(Equation 6)

ΔE_{C}=[E_{C}_total{(Li_{48-z}Mg₁Ni₁Co₄₆O₉₆)+z×E_atom(Li)+2×E_atom(Co)}−{E_total(Li₄₈Co₄₈O₉₆)+E_atom(Mg)+E_atom(Ni)} (Formula 6)

Symbols in Formula 4 to Formula 6 are as follows. Descriptions of symbols which are the same as those in Formula 1 to Formula 3 are omitted.

ΔE_{C}: the stabilization energy when cation occupancy of Li sites is changed.
E_{C}_total (Li_{46-z}Mg₁Ni₁Co₄₈O₉₆): in a LiCoO₂ crystal (192 atoms in total), the energy of the model in which two Li are substituted by one Mg and one Ni and z Li is/are extracted. Note that z is an integer satisfying 0≤z≤46.
E_{C}_total (Li_{47-z}Mg₁Ni₁Co₄₇O₉₆): in a LiCoO₂ crystal (192 atoms in total), the energy of the model in which one Li is substituted by one Mg and one Co is substituted by one Ni. Note that z is an integer satisfying 0≤z≤47.
E_{C}_total (Li_{48-z}Mg₁Ni₁Co₄₆O₉₆): in a LiCoO₂ crystal (192 atoms in total), the energy of the model in which two Co are substituted by one Mg and one Ni and z Li is/are extracted. Note that z is an integer satisfying 0≤z≤48.

In Formulae 4 to 6, z means an extracted Li number. When the value of z is changed, it means that the cation occupancy of Li sites in a lithium composite oxide crystal is changed. In each model, ΔE_{C} is calculated with respect to z, and ΔE_{C} is plotted with respect to the cation occupancy of Li sites or z, whereby the stabilization energy during charging can be estimated.

As shown in FIG. 1, a LiCoO₂ crystal has a structure in which Li ions and Co ions are alternately stacked. Thus, as cations in Li sites decreases, the Van der Waals force between Co layers has stronger influence on the energy of the crystal models (E_{C}_total (Li_{46-z}Mg₁Ni₁Co₄₈O₉₆), E_{C}_total (Li_{47-z}Mg₁Ni₁Co₄₇O₉₆), and E_{C}_total (Li_{48-z}Mg₁Ni₁Co₄₆O₉₆)). Thus, a functional considering the Van der Waals force is preferably used to calculate ΔE_{C}; the above GGA+U(DFT-D2) can be suitably used to calculate ΔE_{C}. As other functionals, OPTB88, DFT-D3, DFT-TS, and the like can be used. A functional which can be used for one embodiment of the present invention is not limited thereto.

The cation occupancy is preferably changed at least between more than or equal to 80% and less than or equal to 100% to plot ΔE_{C}. The cation occupancy is preferably changed between more than or equal to 50% and less than or equal to 100%, more preferably between more than or equal to 0% and less than or equal to 100%. With these conditions, events occurring during charging can be identified.

By making the plot, a crystal structure of a lithium composite oxide can be analyzed through the aforementioned ΔE and ΔE_{C}. In the above example, the positions of a Ni atom and a Mg atom when Mg and Ni are added to LiCoO₂ can be analyzed.

In Formula 4 to Formula 6, z is changed to perform calculation; a plurality of calculations (48 ways at maximum in the above example) on one model is required, which may increase calculation cost. On the other hand, ΔE calculated with Formula 1 to Formula 3 requires a small number of relations between a Mg atom and a Ni atom (approximately up to the fourth proximity) considered with respect to one model, which needs low calculation cost. Thus, ΔE is calculated and appropriate models are selected; then ΔE_{C} of the appropriate model is calculated to make the plot, whereby a crystal structure or substitution positions of a Ni atom and a Mg atom can be analyzed efficiently.

It is expected that a graph showing a relation in which ΔE_{C} increases as z increases is obtained when ΔE_{C} is plotted with respect to the cation occupancy of Li sites or z. When graphs of a plurality of models are made, the magnitude relation between the models is reversed depending on z. For example, with the calculation results in which ΔE_{C} of a model A is smaller than ΔE_{C} of a model B in the range of the cation occupancy of more than or equal to 95% and less than or equal to 100%, whereas ΔE_{C} of the model A is larger than ΔE_{C} of the model B in the range of the cation occupancy of more than or equal to 0% and less than 95%, Li_{(1-x-y)}Co_{(1-a-b)}Ni₍ₓ₊ₐ₎Mg_{(y+b)}O₂ presumably has the structure of the model A when the cation occupancy is more than or equal to 95% and less than or equal to 100%, and has the structure of the model B when the cation occupancy is more than or equal to 0% and less than 95%. In other words, a charging presumably changes the crystal structure. The event caused by charging is preferably analyzed with the following measurement.

### [Measurement of Charge-Discharge Efficiency in First Cycle and Charge-Discharge Efficiency in Second Cycle]

The charge-discharge efficiency of a secondary battery shows approximately 100% when appropriate electrodes are selected, unless materials such as electrode materials and an electrolyte deteriorate, short-circuits occur, and metal lithium precipitates, for example. When a deterioration factor described above does not exist and charge-discharge efficiency falls short of 100%, a crystal structure change of a positive electrode during charging or discharging material can be estimated. Charge-discharge efficiency is calculated from the ratio of a discharging capacity to a charging capacity; when energy supplied by charging is used for something other than Li extraction, charge-discharge efficiency decreases. When a crystal model of a positive electrode material changes, decrease of charge-discharge efficiency is observed. Thus, change of a crystal model of a lithium composite oxide can be analyzed by measuring charge-discharge efficiency.

To analyze change of a crystal model accurately, the deterioration factors are preferably few. Thus, the charge-discharge efficiency in the first cycle is preferably measured. When a crystal model changes, events occurring in a crystal can be analyzed in detail by analyzing whether the change is reversible or irreversible. Thus, the charge-discharge efficiency in the second cycle is more preferably measured in addition to the charge-discharge efficiency in the first cycle. When the second measurement shows a similar result of the first measurement, events occurring in a crystal are presumably reversible. On the other hand, when the second measurement shows a different result from the first measurement, for example, the charge-discharge efficiency in the first cycle is less than 100% and the charge-discharge efficiency in the second cycle is 100%, the events occurred in a crystal are presumably irreversible and occurred only at the first cycle. Analysis can be conducted in more detail by comparing the charge-discharge efficiency in the first cycle and that in the second cycle.

If deterioration factors are few, the charge-discharge efficiency in the third and the subsequent cycles can be compared with that in the first cycle. Charge-discharge efficiency in any cycle can be compared such as the second and after the third cycle. When events occurring in a crystal are irreversible, the events may be observed only at the first cycle. Thus, the charge-discharge efficiency in the first cycle is preferably measured. Considering deterioration factors, the charge-discharge efficiency in the first cycle and that in the second cycle are more preferably compared.

To measure the charge-discharge efficiency of a compound represented by the chemical formula Li_{(1-x-y)}Co_{(1-a-b)}Ni₍ₓ₊ₐ₎Mg_{(y+b)}O₂, it is necessary to synthesize the compound firstly. Then, the synthesized compound is used to make a battery cell and the charge-discharge efficiency of the battery cell is measured. Measuring charge-discharge efficiency needs these steps. When measurements such as an XRD measurement, which do not relate to charging, discharging, an electrolyte, and the like, are conducted on the compound represented by the chemical formula Li_{(1-x-y)}Co_{(1-a-b)}Ni₍ₓ₊ₐ₎Mg_{(y+b)}O₂, a battery cell need not be made. When the measurement relating to a compound represented by the chemical formula Li_{(1-x-y)}Co_{(1-a-b)}Ni₍ₓ₊ₐ₎Mg_{(y+b)}O₂ is conducted, at least a step to synthesize the compound and a step to measure the characteristics of the compound are needed. To measure charge-discharge efficiency, a step to make a battery cell is also needed. Other steps may be included for measurement.

Analysis is conducted with a combination of ΔE, ΔE_{C}, and measurement of charge-discharge efficiency described in this embodiment, whereby the crystal structure and substitution positions of a lithium composite oxide can be accurately analyzed. A plurality of calculation methods is used to perform calculation efficiently and analysis is conducted with a combination of measurements; these are the features of this invention. Calculation and measurement are combined, which makes it possible to evaluate the validity of a calculation result and events expected from the calculation result; detail analysis can be conducted. A compound represented by the chemical formula Li_{(1-x-y)}Co_{(1-a-b)}Ni₍ₓ₊ₐ₎Mg_{(y+b)}O₂ has especially good characteristics in the ranges of 0<x+a≤0.015 and 0<y+b≤0.06; however, the crystal structure thereof is difficult to analyze in detail only with measurement since Mg and Ni are slightly contained. Thus, the analysis method of one embodiment of the present invention can be suitably used. Based on an analysis result from calculation and measurement, another analysis with calculation and measurement is conducted, whereby more accurate analysis can be conducted. The calculations and measurements shown in this embodiment can be repeated. Based on the results from the calculations and the measurements shown in this embodiment, an additional analysis can be conducted.

A measurement of charge-discharge efficiency is described as an example of measurement; a dQ/dV measurement, an XRD measurement, a magnetization measurement, a Li NMR measurement, and the like can be used as the measurements of one embodiment of the present invention. The measurements of one embodiment of the present invention are not limited to them.

This embodiment can be implemented in appropriate combination with any of the other embodiments.

### Embodiment 2

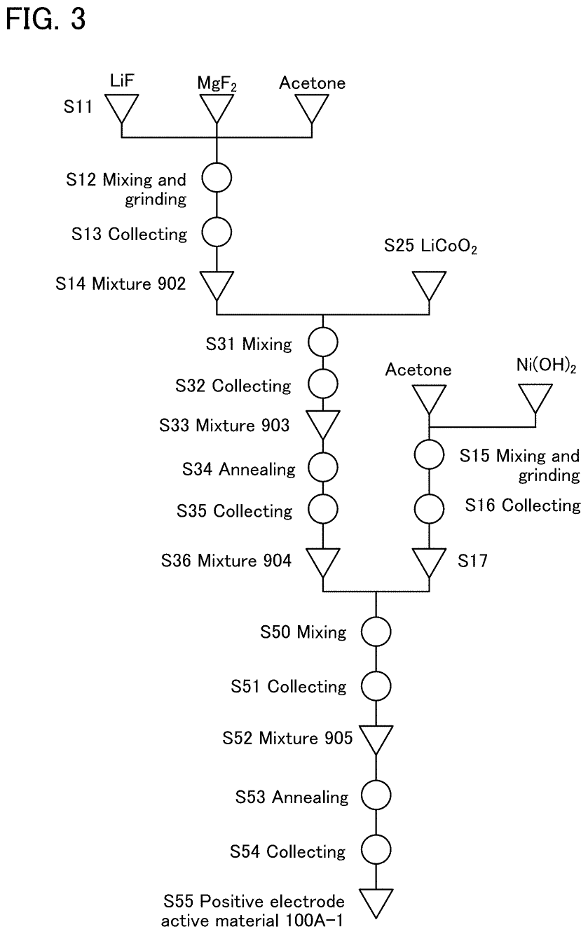

An example of a method to make a compound represented by the chemical formula Li_{(1-x-y)}Co_{(1-a-b)}Ni₍ₓ₊ₐ₎Mg_{(y+b)}O₂ is described using FIG. 3.

As shown in Step S11 in FIG. 3, lithium fluoride that is a fluorine source and magnesium fluoride that is a magnesium source are first prepared as materials of the mixture 902. Among them, lithium fluoride, which has a relatively low melting point of 848° C., is preferable because it is easily melted in an annealing process described later. Lithium fluoride can be used as both the lithium source and the fluorine source. Magnesium fluoride can be used as both the fluorine source and the magnesium source.

In this embodiment, lithium fluoride LiF is prepared as the fluorine source and the lithium source, and magnesium fluoride MgF₂ is prepared as the fluorine source and the magnesium source (Step S11 in FIG. 3). The molar ratio of lithium fluoride LiF to magnesium fluoride MgF₂ is preferably LiF:MgF₂=u:1 (0≤u≤1.9), further preferably LiF:MgF₂=u:1 (0.1≤u≤0.5), still further preferably LiF:MgF₂=u:1 (u=the vicinity of 0.33).

In addition, in the case where the following mixing and grinding steps are performed by a wet process, a solvent is prepared. As the solvent, ketone such as acetone; alcohol such as ethanol or isopropanol; ether; dioxane; acetonitrile; N-methyl-2-pyrrolidone (NMP); or the like can be used. An aprotic solvent that hardly reacts with lithium is further preferably used. In this embodiment, acetone is used (see Step S11 in FIG. 3).

Next, the materials of the mixture 902 are mixed and ground (Step S12 in FIG. 3). The mixing can be performed by a dry process or a wet process; the wet process is preferable because the materials can be ground to the smaller size. For example, a ball mill, a bead mill, or the like can be used for the mixing. When the ball mill is used, a zirconia ball is preferably used as media, for example. The mixing step and the grinding step are preferably performed sufficiently to pulverize the mixture 902.

The materials mixed and ground in the above are collected (Step S13 in FIG. 3), whereby the mixture 902 is obtained (Step S14 in FIG. 3).

For example, D50 of the mixture 902 is preferably greater than or equal to 600 nm and less than or equal to 20 μm, further preferably greater than or equal to 1 μm and less than or equal to 10 μm. When mixed with a composite oxide containing lithium, a transition metal, and oxygen in the later step, the mixture 902 pulverized to such a small size is easily attached to surfaces of composite oxide particles uniformly. The mixture 902 is preferably attached to the surfaces of the composite oxide particles uniformly because both halogen and magnesium are easily distributed to the superficial portion of the composite oxide particles after heating. When there is a region containing neither halogen nor magnesium in the superficial portion, the above-described pseudo-spinel crystal structure in the charged state is less likely to be formed.

Next, a lithium source is prepared as shown in Step S25. A composite oxide containing lithium, a transition metal, and oxygen that is synthesized in advance may be used as Step S25.

For example, as lithium cobaltate synthesized in advance, a lithium cobaltate particle (product name: CELLSEED C-10N) manufactured by NIPPON CHEMICAL INDUSTRIAL CO., LTD. can be used. This is lithium cobaltate in which the average particle diameter (D50) is approximately 12 μm, and in the impurity analysis by a glow discharge mass spectroscopy method (GD-MS), the magnesium concentration and the fluorine concentration are less than or equal to 50 ppm wt, the calcium concentration, the aluminum concentration, and the silicon concentration are less than or equal to 100 ppm wt, the nickel concentration is less than or equal to 150 ppm wt, the sulfur concentration is less than or equal to 500 ppm wt, the arsenic concentration is less than or equal to 1100 ppm wt, and the concentrations of elements other than lithium, cobalt, and oxygen are less than or equal to 150 ppm wt.

The composite oxide including lithium, the transition metal, and oxygen in Step S25 preferably has a layered rock-salt crystal structure with few defects and distortions. Therefore, the composite oxide is preferably a composite oxide with few impurities. In the case where the composite oxide including lithium, the transition metal, and oxygen includes a lot of impurities, the crystal structure is highly likely to have a lot of defects or distortions.

Next, the mixture 902 and the composite oxide including lithium, the transition metal, and oxygen are mixed (Step S31 in FIG. 3). The atomic ratio of the transition metal TM in the composite oxide containing lithium, the transition metal, and oxygen to magnesium MgMix1 contained in the mixture 902 is preferably TM:MgMix1=1:v(0.005≤v≤0.05), further preferably TM:MgMix1=1:v(0.007≤v≤0.04), still further preferably approximately TM:MgMix1=1:0.02.

The condition of the mixing in Step S31 is preferably milder than that of the mixing in Step S12 not to damage the particles of the composite oxide. For example, a condition with a lower rotation frequency or shorter time than the mixing in Step S12 is preferable. In addition, it can be said that the dry process has a milder condition than the wet process. For example, a ball mill, a bead mill, or the like can be used for the mixing. When the ball mill is used, a zirconia ball is preferably used as media, for example.

The materials mixed in the above are collected (Step S32 in FIG. 3), whereby a mixture 903 is obtained (Step S33 in FIG. 3).

Next, the mixture 903 is heated (Step S34 in FIG. 3). This step is referred to as annealing or second heating in some cases to distinguish this step from the heating step performed before.

The annealing is preferably performed at an appropriate temperature for an appropriate time. The appropriate temperature and time depend on the conditions such as the particle size and the composition of the composite oxide including lithium, the transition metal, and oxygen in Step S25. In the case where the particle size is small, the annealing is preferably performed at a lower temperature or for a shorter time than the case where the particle size is large, in some cases.

When the average particle diameter (D50) of the particles in Step S25 is approximately 12 μm, for example, an annealing temperature is preferably higher than or equal to 600° C. and lower than or equal to 950° C., for example. The annealing time is preferably longer than or equal to 3 hours, further preferably longer than or equal to 10 hours, still further preferably longer than or equal to 60 hours, for example.

On the other hand, when the average particle diameter (D50) of the particles in Step S25 is approximately 5 μm, the annealing temperature is preferably higher than or equal to 600° C. and lower than or equal to 950° C., for example. The annealing time is preferably longer than or equal to 1 hour and shorter than or equal to 10 hours, further preferably approximately 2 hours, for example.

The temperature decreasing time after the annealing is, for example, preferably longer than or equal to 10 hours and shorter than or equal to 50 hours.

It is considered that when the mixture 903 is annealed, a material having a low melting point (e.g., lithium fluoride, which has a melting point of 848° C.) in the mixture 902 is melted first and distributed to the superficial portion of the composite oxide particle. Next, the existence of the melted material decreases the melting points of other materials, presumably resulting in melting of the other materials. For example, magnesium fluoride (melting point: 1263° C.) is presumably melted and distributed to the superficial portion of the composite oxide particle.

The elements included in the mixture 903 are diffused faster in the superficial portion and the vicinity of the grain boundary than inside the composite oxide particles. Therefore, the concentrations of magnesium and halogen in the superficial portion and the vicinity of the grain boundary are higher than those of magnesium and halogen inside the composite oxide particles.

The materials annealed in the above manner are collected (Step S35 in FIG. 3), whereby a mixture 904 is obtained (Step S36 in FIG. 3).

Next, as shown in Step S50, the mixture 904 and pulverized nickel hydroxide are mixed. Then, the mixed materials are collected (Step S51). The pulverized nickel hydroxide is formed in advance by Step S15 for mixing nickel hydroxide and acetone and Step S16 for collecting the mixture. Through Step S16, the pulverized nickel hydroxide is obtained (Step S17).

The materials mixed in Step S50 are collected in Step S51, whereby a mixture 905 is obtained (Step S52 in FIG. 3).

Then, the obtained mixture is heated (Step S53 in FIG. 3).

As for the heating time, the time for keeping the heating temperature within a predetermined range is preferably longer than or equal to 1 hour and shorter than or equal to 80 hours.

The heating temperature is lower than 1000° C., preferably higher than or equal to 700° C. and lower than or equal to 950° C., further preferably approximately 850° C.

The heating is preferably performed in an oxygen-containing atmosphere.

In this embodiment, the heating temperature is 850° C. and kept for 2 hours, the temperature rising rate is 200° C./h, and the flow rate of oxygen is 10 L/min.

Here, the heating temperature in Step S53 is preferably lower than the heating temperature in Step S34.

### <Step S54 and Step S55>

Next, cooled particles are collected (Step S54 in FIG. 3). Moreover, the particles are preferably filtered. Through the above steps, a positive electrode active material 100A-1, which is an example of a compound represented by the chemical formula Li_{(1-x-y)}Co_{(1-a-b)}Ni₍ₓ₊ₐ₎Mg_{(y+b)}O₂, can be made (Step S55 in FIG. 3).

The positive electrode active material 100A-1 obtained by the above manufacturing method is described.

### [Structure of Positive Electrode Active Material]

A material with a layered rock-salt crystal structure, such as lithium cobaltate (LiCoO₂), is known to have a high discharge capacity and excel as a positive electrode active material of a secondary battery. As an example of the material with a layered rock-salt crystal structure, a composite oxide represented by LiMO₂ is given. As an example of the element M, one or more elements selected from Co and Ni can be given. As another example of the element M, in addition to one or more elements selected from Co and Ni, one or more elements selected from Al and Mg can be given.

It is known that the Jahn-Teller effect in a transition metal compound varies in degree according to the number of electrons in the d orbital of the transition metal.

In a compound containing nickel, distortion is likely to be caused because of the Jahn-Teller effect in some cases. Accordingly, when high-voltage charging and discharging are performed on LiNiO₂, the crystal structure might be disordered because of the distortion. The influence of the Jahn-Teller effect is suggested to be small in LiCoO₂; hence, LiCoO₂ is preferable because the resistance to high-voltage charging and discharging is higher in some cases.

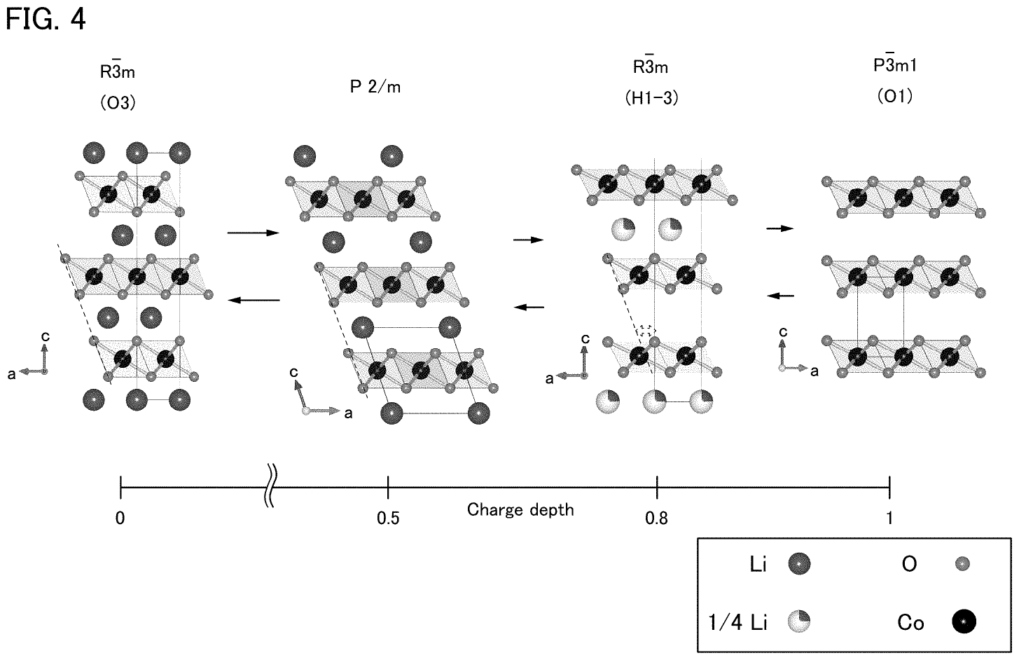

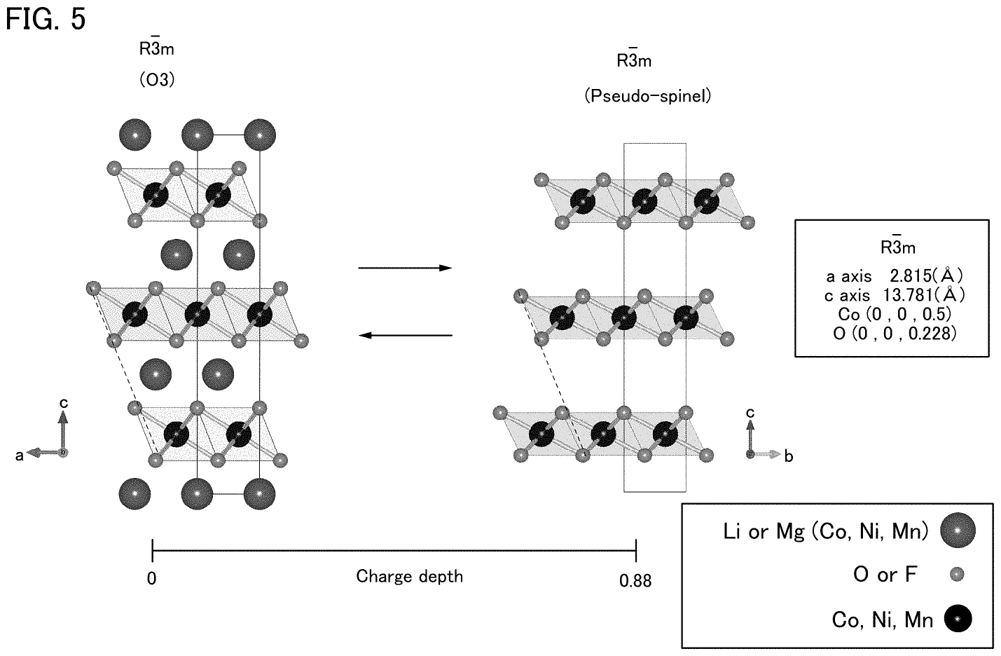

Positive electrode active materials are described with reference to FIG. 4 and FIG. 5. In FIG. 4 and FIG. 5, the case where cobalt is used as a transition metal contained in the positive electrode active material is described.

A compound represented by the chemical formula Li_{(1-x-y)}Co_{(1-a-b)}Ni₍ₓ₊ₐ₎Mg_{(y+b)}O₂ can have small difference of CoO₂ layers through repetitions of high-voltage charging and discharging. Furthermore, change in volume can be small. Thus, the compound can achieve excellent cycle performances. In addition, the compound can have a stable crystal structure in a high-voltage charged state. Thus, in the compound, a short circuit is less likely to occur while the high-voltage charged state is maintained. This is preferable because the safety is further improved. It is particularly preferable to satisfy 0<x+a≤0.015 and 0<y+b≤0.06, in which case the compound shows good performances.

In the compound, there is a small difference in change in the crystal structure and volume in comparison with the same number of transition metal atoms between a sufficiently discharged state and a high-voltage charged state.

FIG. 5 shows the crystal structures of the positive electrode active material 100A-1 before and after being charged and discharged. The positive electrode active material 100A-1 is a composite oxide represented by the chemical formula Li_{(1-x-y)}Co_{(1-a-b)}Ni₍ₓ₊ₐ₎Mg_{(y+b)}O₂.

The crystal structure with a charge depth of 0 (discharged state) in FIG. 5 is R-3m (O3), which is the same as that in FIG. 4. Meanwhile, the positive electrode active material 100A-1 with a charge depth in a sufficiently charged state includes a crystal whose structure is different from the H1-3 type structure. This structure belongs to the space group R-3m, and is not a spinel crystal structure but a structure in which an ion of cobalt, magnesium, or the like is coordinated to six oxygen atoms and the cation arrangement has symmetry similar to that of the spinel crystal structure. This structure is thus referred to as the pseudo-spinel crystal structure in this specification and the like. Note that although the indication of lithium is omitted in the diagram of the pseudo-spinel crystal structure shown in FIG. 5 to explain the symmetry of cobalt atoms and the symmetry of oxygen atoms, a lithium of 20 atomic % or less, for example, with respect to cobalt practically exists between the CoO₂ layers. In addition, in both the O3-type crystal structure and the pseudo-spinel crystal structure, a slight amount of magnesium preferably exists between the CoO₂ layers, i.e., in lithium sites. In addition, a slight amount of halogen such as fluorine may exist in oxygen sites at random.

Note that in the pseudo-spinel crystal structure, oxygen is tetracoordinated to a light element such as lithium in some cases. Also in that case, the ion arrangement has symmetry similar to that of the spinel crystal structure.

The pseudo-spinel crystal structure can also be regarded as a crystal structure that contains Li between layers at random but is similar to a CdCl₂ type crystal structure. The crystal structure similar to the CdCl₂ type crystal structure is close to a crystal structure of lithium nickel oxide when charged up to a charge depth of 0.94 (Li_{0.06}NiO₂); however, pure lithium cobaltate or a layered rock-salt positive electrode active material containing a large amount of cobalt is known not to have this crystal structure generally.

Anions of a layered rock-salt crystal and anions of a rock-salt crystal have cubic closest packed structures (face-centered cubic lattice structures). Anions of a pseudo-spinel crystal are also presumed to have cubic closest packed structures. When the pseudo-spinel crystal is in contact with the layered rock-salt crystal and the rock-salt crystal, there is a crystal plane at which orientations of cubic closest packed structures composed of anions are aligned. Note that a space group of the layered rock-salt crystal and the pseudo-spinel crystal is R-3m, which is different from a space group Fm-3m of a rock-salt crystal (a space group of a general rock-salt crystal) and a space group Fd-3m of a rock-salt crystal (a space group of a rock-salt crystal having the simplest symmetry); thus, the Miller index of the crystal plane satisfying the above conditions in the layered rock-salt crystal and the pseudo-spinel crystal is different from that in the rock-salt crystal. In this specification, a state where the orientations of the cubic closest packed structures composed of anions in the layered rock-salt crystal, the pseudo-spinel crystal, and the rock-salt crystal are aligned is referred to as a state where crystal orientations are substantially aligned in some cases.

In the positive electrode active material 100A-1, a change in the crystal structure when the positive electrode active material 100A-1 is charged with a high voltage and a large amount of lithium is extracted is inhibited as compared with the positive electrode active material 100C. As indicated by the dotted lines in FIG. 4, for example, there is a very little shift in the CoO₂ layers between the crystal structures.

More specifically, the structure of the positive electrode active material 100A-1 is highly stable even when a charging voltage is high. For example, at a charge voltage that makes the positive electrode active material 100C have the H1-3 type crystal structure, for example, at a voltage of approximately 4.6 V with reference to the potential of lithium metal, the positive electrode active material 100A-1 can have the R-3m (O3) crystal structure. Moreover, in a higher charge voltage region, for example, at voltages of approximately 4.65 V to 4.7 V with reference to the potential of lithium metal, the pseudo-spinel crystal structure can be obtained. At a much higher charging voltage, the H1-3 type crystal is eventually observed in some cases. In the case where graphite, for instance, is used as a negative electrode active material in a secondary battery, when the voltage of the secondary battery ranges from 4.3 V to 4.5 V, for example, the R-3m (O3) crystal structure can be maintained. In a higher charging voltage region, for example, at voltages of 4.35 V to 4.55 V with reference to the potential of lithium metal, the pseudo-spinel crystal structure can be obtained.

Thus, in the positive electrode active material 100A-1, the crystal structure is less likely to be disordered even when charging and discharging are repeated at a high voltage.

Note that in the unit cell of the pseudo-spinel crystal structure, coordinates of cobalt and oxygen can be represented by Co (0, 0, 0.5) and O (0, 0, x) within the range of 0.20≤x≤0.25.

A slight amount of magnesium existing between the CoO₂ layers, i.e., in lithium sites at random, has an effect of inhibiting a difference in the CoO₂ layers. Thus, the existence of magnesium between the CoO₂ layers makes it easier to obtain the pseudo-spinel crystal structure. Therefore, magnesium is preferably distributed in the entire particle of the positive electrode active material 100A-1. In addition, to distribute magnesium in the entire particle, heat treatment is preferably performed in the formation process of the positive electrode active material 100A-1.

However, cation mixing occurs when the heat treatment temperature is excessively high, so that magnesium is highly likely to enter the cobalt sites. Magnesium in the cobalt sites eliminates the effect of maintaining the R-3m structure. Furthermore, when the heat treatment temperature is excessively high, adverse effects such as reduction of cobalt to be divalent and transpiration of lithium are concerned.

In view of the above, a halogen compound such as a fluorine compound is preferably added to lithium cobaltate before the heat treatment for distributing magnesium in the entire particle. The addition of the halogen compound decreases the melting point of lithium cobaltate. The decrease in the melting point makes it easier to distribute magnesium in the entire particle at a temperature at which the cation mixing is unlikely to occur. Furthermore, when the fluorine compound exists, it is expected that the corrosion resistance to hydrofluoric acid generated by decomposition of an electrolyte solution is improved.

When the magnesium concentration is higher than a predetermined value, the effect of stabilizing a crystal structure becomes small in some cases. This is probably because magnesium enters the cobalt sites in addition to the lithium sites. The number of magnesium atoms in the positive electrode active material of one embodiment of the present invention is preferably 0.001 to 0.1 times, preferably larger than 0.01 times and less than 0.04 times, still further preferably approximately 0.02 times the number of cobalt atoms. The magnesium concentration described here may be a value obtained by element analysis on the entire particle of the positive electrode active material using ICP-MS or the like, or may be a value based on the ratio of the raw materials mixed in the formation process of the positive electrode active material, for example.

The number of nickel atoms in the positive electrode active material 100A-1 of one embodiment of the present invention is preferably 7.5% or less, further preferably 0.05% to 40%, still further preferably 0.1% to 2% of the number of cobalt atoms. The nickel concentration described here may be a value obtained by element analysis on the entire particle of the positive electrode active material using ICP-MS or the like, or may be a value based on the ratio of the raw materials mixed in the process of forming the positive electrode active material, for example.

The detail structure of the positive electrode active material 100A-1 can be analyzed with the analysis methods shown in Embodiment 1.

### «Particle Size»

A too large particle size of the positive electrode active material 100A-1 causes problems such as difficulty in lithium diffusion and too much surface roughness of an active material layer in coating to a current collector. By contrast, a too small particle size causes problems such as difficulty in loading the active material layer in coating to the current collector and overreaction with an electrolyte solution. Therefore, an average particle size (D50, also referred to as median diameter) is preferably more than or equal to 1 μm and less than or equal to 100 μm, further preferably more than or equal to 2 μm and less than or equal to 40 μm, still further preferably more than or equal to 5 μm and less than or equal to 30 μm.

### <Analysis Method>

Whether or not a positive electrode active material is the positive electrode active material having the pseudo-spinel crystal structure when charged with a high voltage can be determined by analyzing a high-voltage charged positive electrode using XRD, electron diffraction, neutron diffraction, electron spin resonance (ESR), nuclear magnetic resonance (NMR), or the like. The XRD is particularly preferable because the symmetry of a transition metal such as cobalt contained in the positive electrode active material can be analyzed with high resolution, the degrees of crystallinity and the crystal orientations can be compared, the distortion of lattice periodicity and the crystallite size can be analyzed, and a positive electrode obtained by disassembling a secondary battery can be measured without any change with sufficient accuracy, for example.

As described so far, the positive electrode active material 100A-1 has a feature of a small change in the crystal structure between the high-voltage charged state and the discharged state. A material where 50 wt % or more of the crystal structure greatly changes between the high-voltage charged state and the discharged state is not preferable because the material cannot withstand a high-voltage charging and discharging. In addition, it should be noted that a target crystal structure is not obtained in some cases only by adding impurity elements. For example, although the positive electrode active material that is lithium cobaltate containing magnesium and fluorine is a commonality, the positive electrode active material has 60 wt % or more of the pseudo-spinel crystal structure in some cases, and has 50 wt % or more of the H1-3 type crystal structure in other cases, when charged with a high voltage. Furthermore, at a predetermined voltage, the positive electrode active material has almost 100 wt % of the pseudo-spinel crystal structure, and with an increase in the predetermined voltage, the H1-3 type crystal structure is generated in some cases. The crystal structure of the positive electrode active material 100A-1 is preferably analyzed with XRD or the like. It is more preferable to analyze it with the analysis methods described in Embodiment 1. The combination of the analysis methods and measurement such as XRD enables more detail analysis.

Note that a positive electrode active material in the high-voltage charged state or the discharged state sometimes causes a change in the crystal structure when exposed to the air. For example, the pseudo-spinel crystal structure changes into the H1-3 type crystal structure in some cases. Thus, all samples are preferably handled in an inert atmosphere such as an argon atmosphere.

### <Positive Electrode Active Material (LiCoO₂) of Reference>

A positive electrode active material (lithium cobaltate) shown in FIG. 4 is lithium cobaltate (LiCoO₂) to which halogen and magnesium are not added in a manufacturing method described later. As described in Non-Patent Document 1, Non-Patent Document 2, and the like, the crystal structure of lithium cobaltate shown in FIG. 4 changes depending on the charge depth.

As shown in FIG. 4, lithium cobaltate with a charge depth of 0 (discharged state) includes a region having the crystal structure of the space group R-3m, and includes three CoO₂ layers in a unit cell. Thus, this crystal structure is referred to as an O3-type crystal structure in some cases. Note that the CoO₂ layer has a structure in which octahedral geometry with oxygen atoms hexacoordinated to cobalt continues on a plane in the edge-sharing state.

Furthermore, when the charge depth is 1, LiCoO₂ has the crystal structure of the space group P-3 μml, and one CoO₂ layer exists in a unit cell. Thus, this crystal structure is referred to as an O1-type crystal structure in some cases.

Moreover, lithium cobaltate when the charge depth is approximately 0.88 has the crystal structure of the space group R-3m. This structure can also be regarded as a structure in which CoO₂ structures such as P-3 μml (O1) and LiCoO₂ structures such as R-3m (O3) are alternately stacked. Thus, this crystal structure is referred to as an H1-3 type crystal structure in some cases. Note that the number of cobalt atoms per unit cell in the actual H1-3 type crystal structure is twice as large as that of cobalt atoms per unit cell in other structures. However, in this specification including FIG. 5, the c-axis of the H1-3 type crystal structure is described half that of the unit cell for easy comparison with the other structures.

For the H1-3 type crystal structure, as disclosed in Non-Patent Document 3, the coordinates of cobalt and oxygen in the unit cell can be expressed as follows, for example: Co (0, 0, 0.42150±0.00016), O₁ (0, 0, 0.27671±0.00045), and O₂ (0, 0, 0.11535±0.00045). Note that O₁ and O₂ are each an oxygen atom. In this manner, the H1-3 type crystal structure is represented by a unit cell including one cobalt and two oxygen. Meanwhile, the pseudo-spinel crystal structure of one embodiment of the present invention shown in FIG. 5 is preferably represented by a unit cell including one cobalt and one oxygen, as described later. This means that the symmetry of cobalt and oxygen differs between the pseudo-spinel structure and the H1-3 type structure, and the amount of change from the O3 structure is smaller in the pseudo-spinel structure than in the H1-3 type structure. A preferred unit cell for representing a crystal structure in a positive electrode active material is selected such that the value of GOF (good of fitness) is smaller in the Rietveld analysis of XRD patterns, for example.

When charging with a high voltage of 4.6 V or higher based on the redox potential of a lithium metal or charging with a large charge depth of 0.8 or more and discharging are repeated, the crystal structure of lithium cobaltate changes (i.e., a non-equilibrium phase change occurs) repeatedly between the H1-3 type crystal structure and the R-3m (O3) structure in a discharged state.

However, there is a large difference in the position of the CoO₂ layer between these two crystal structures. As indicated by the dotted lines and the arrows in FIG. 4, the CoO₂ layer in the H1-3 type crystal structure greatly differs from that in R-3m (O3). Such a dynamic structural change might adversely affect the stability of the crystal structure.

A difference in volume is also large. A difference in volume in comparison with the same number of cobalt atoms between the H1-3 type crystal structure and the O3-type crystal structure in the discharged state is 3.0% or more.

In addition, a structure in which CoO₂ layers are continuous, such as P-3 μml (O1), included in the H1-3 type crystal structure is highly likely to be unstable.

Thus, the repeated high-voltage charging and discharging break the crystal structure of lithium cobaltate. The break of the crystal structure degrades the cycle performance. This is probably because the break of the crystal structure reduces sites where lithium can stably exist and makes it difficult to insert and extract lithium.

### Embodiment 3

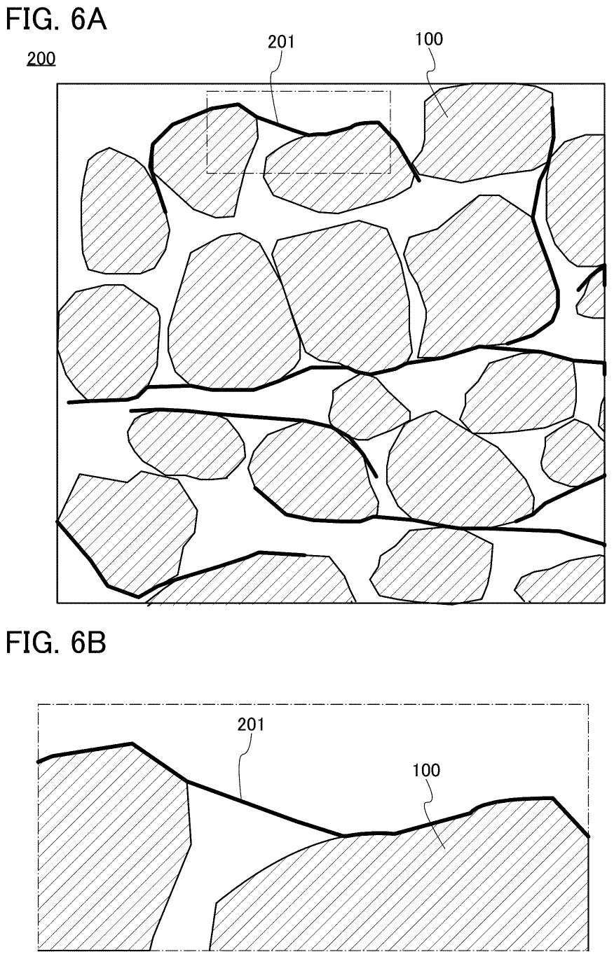

In this embodiment, examples of materials that can be used for a secondary battery containing the positive electrode active material 100 described in FIG. 6A and FIG. 6B are described. In this embodiment, a secondary battery in which a positive electrode, a negative electrode, and an electrolyte solution are wrapped in an exterior body is described as an example. As the positive electrode active material 100, a compound represented by the chemical formula Li_{(1-x-y)}Co_{(1-a-b)}Ni₍ₓ₊ₐ₎Mg_{(y+b)}O₂ and the positive electrode active material 100A-1 described in the above embodiments can be used.

### [Positive Electrode]

The positive electrode includes a positive electrode active material layer and a positive electrode current collector.

### <Positive Electrode Active Material Layer>

The positive electrode active material layer includes a positive electrode active material particle. The positive electrode active material layer may contain a conductive additive and a binder.

As the positive electrode active material particle, the positive electrode active material 100 can be used. As the positive electrode active material 100, the lithium composite oxide described in the above embodiment, such as a compound represented by the chemical formula Li_{(1-x-y)}Co_{(1-a-b)}Ni₍ₓ₊ₐ₎Mg_{(y+b)}O₂ and the positive electrode active material 100A-1, can be used. In the chemical formula, it is preferable to satisfy 0<x+a≤0.015 and 0<y+b≤0.06. A secondary battery with the compound hardly deteriorates and thus has good safety.

Examples of the conductive additive include a carbon material, a metal material, and a conductive ceramic material. Alternatively, a fiber material may be used as the conductive additive. The content of the conductive additive to the total amount of the active material layer is preferably greater than or equal to 1 wt % and less than or equal to 10 wt %, further preferably greater than or equal to 1 wt % and less than or equal to 5 wt %.

A network for electric conduction can be formed in the electrode by the conductive additive. The conductive additive also allows the maintenance of a path for electric conduction between the positive electrode active materials. The addition of the conductive additive to the active material layer increases the electric conductivity of the active material layer.

Examples of the conductive additive include natural graphite, artificial graphite such as mesocarbon microbeads, and carbon fiber. As carbon fiber, mesophase pitch-based carbon fiber and isotropic pitch-based carbon fiber can be used. Furthermore, as carbon fiber, carbon nanofiber and carbon nanotube can be used. Carbon nanotube can be formed by, for example, a vapor deposition method. Other examples of the conductive additive include carbon materials such as carbon black (e.g., acetylene black (AB)), graphite (black lead) particles, graphene, and fullerene. Alternatively, metal powder or metal fibers of copper, nickel, aluminum, silver, gold, or the like, a conductive ceramic material, or the like can be used.

Alternatively, a graphene compound may be used as the conductive additive.

A graphene compound has excellent electrical characteristics of high conductivity and excellent physical properties of high flexibility and high mechanical strength in some cases. Furthermore, a graphene compound has a planar shape. A graphene compound enables low-resistance surface contact. Furthermore, a graphene compound has extremely high conductivity even with a small thickness in some cases and thus allows a conductive path to be formed in an active material layer efficiently even with a small amount. Thus, a graphene compound is preferably used as the conductive additive, in which case the area where the active material and the conductive additive are in contact with each other can be increased. In addition, the graphene compound is preferable because electrical resistance can be reduced in some cases. Here, it is particularly preferable to use, for example, graphene, multilayer graphene, graphene quantum dot, or reduced graphene oxide (hereinafter, RGO) as a graphene compound. Note that RGO refers to a compound obtained by reducing graphene oxide (GO), for example.

In the case where an active material particle with a small particle diameter (e.g., 1 μm or less) is used, the specific surface area of the active material particle is large and thus more conductive paths for connecting the active material particles are needed. In such a case, a graphene compound that can efficiently form a conductive path even in a small amount is particularly preferably used.

A cross-sectional structure example of an active material layer 200 containing a graphene compound as a conductive additive is described below.

FIG. 6A is a longitudinal cross-sectional view of the active material layer 200. The active material layer 200 includes particles of the positive electrode active material 100, a graphene compound 201 serving as a conductive additive, and a binder (not shown). Here, graphene or multilayer graphene may be used as the graphene compound 201, for example. The graphene compound 201 preferably has a sheet-like shape. The graphene compound 201 may have a sheet-like shape formed of a plurality of sheets of multilayer graphene and/or a plurality of sheets of graphene that partly overlap with each other.

The longitudinal cross section of the active material layer 200 in FIG. 6A shows substantially uniform dispersion of the sheet-like graphene compounds 201 in the active material layer 200. The graphene compounds 201 are schematically shown by thick lines in FIG. 6A but are actually thin films with a thickness corresponding to the thickness of a single layer or a multilayer of carbon molecules. A plurality of graphene compounds 201 are formed in such a way as to wrap or cover the plurality of positive electrode active material particles 100 or adhere to the surfaces of the plurality of positive electrode active material particles 100, so that the graphene compounds 201 make surface contact with the positive electrode active material particles 100.

Here, the plurality of graphene compounds are bonded to each other to form a net-like graphene compound sheet (hereinafter, referred to as a graphene compound net or a graphene net). The graphene net covering the active material can function as a binder for bonding active materials. The amount of binder can thus be reduced, or the binder does not have to be used. This can increase the proportion of the active material in the electrode volume or the electrode weight. That is to say, the capacity of the power storage device can be increased.

Here, it is preferable to perform reduction after a layer to be the active material layer 200 is formed in such a manner that graphene oxide is used as the graphene compound 201 and mixed with an active material. When graphene oxide with extremely high dispersibility in a polar solvent is used for the formation of the graphene compounds 201, the graphene compounds 201 can be substantially uniformly dispersed in the active material layer 200. The solvent is removed by volatilization from a dispersion medium containing the uniformly-dispersed graphene oxide to reduce the graphene oxide; hence, the graphene compounds 201 remaining in the active material layer 200 are partly overlap with each other and are dispersed such that surface contact is made, thereby forming a three-dimensional conduction path. Note that graphene oxide can be reduced either by heat treatment or with the use of a reducing agent, for example.

Unlike a particle of conductive additive such as acetylene black, which makes point contact with an active material, the graphene compound 201 is capable of making low-resistance surface contact; accordingly, the electrical conduction between the particles of the positive electrode active material 100 and the graphene compound 201 can be improved with a smaller amount of the graphene compound 201 than that of a normal conductive additive. This can increase the proportion of the positive electrode active material 100 in the active material layer 200. This can increase discharge capacity of the power storage device.

As the binder, a rubber material such as styrene-butadiene rubber (SBR), styrene-isoprene-styrene rubber, acrylonitrile-butadiene rubber, butadiene rubber, or ethylene-propylene-diene copolymer can be used, for example. Alternatively, fluororubber can be used as the binder.

For the binder, for example, water-soluble polymers are preferably used. As the water-soluble polymers, for example, a polysaccharide can be used. As the polysaccharide, for example, a cellulose derivative such as carboxymethyl cellulose (CMC), methyl cellulose, ethyl cellulose, hydroxypropyl cellulose, diacetyl cellulose, and regenerated cellulose or starch can be used. It is further preferred that such water-soluble polymers be used in combination with any of the above rubber materials.

Alternatively, as the binder, a material such as polystyrene, poly(methyl acrylate), poly(methyl methacrylate) (PMMA), sodium polyacrylate, polyvinyl alcohol (PVA), polyethylene oxide (PEO), polypropylene oxide, polyimide, polyvinyl chloride, polytetrafluoroethylene, polyethylene, polypropylene, polyisobutylene, polyethylene terephthalate, nylon, polyvinylidene fluoride (PVDF), polyacrylonitrile (PAN), ethylene-propylene-diene polymer, polyvinyl acetate, or nitrocellulose is preferably used.

A plurality of the above materials may be used in combination for the binder.

For example, a material having a significant viscosity modifying effect and another material may be used in combination. For example, a rubber material or the like has high adhesion or high elasticity but may have difficulty in viscosity modification when mixed in a solvent. In such a case, a rubber material or the like is preferably mixed with a material having a significant viscosity modifying effect, for example. As a material having a significant viscosity modifying effect, for example, a water-soluble polymer is preferably used. An example of a water-soluble polymer having an especially significant viscosity modifying effect is the above-mentioned polysaccharide; for example, a cellulose derivative such as carboxymethyl cellulose (CMC), methyl cellulose, ethyl cellulose, hydroxypropyl cellulose, diacetyl cellulose, or regenerated cellulose, or starch can be used.

Note that a cellulose derivative such as carboxymethyl cellulose obtains a higher solubility when converted into a salt such as a sodium salt or an ammonium salt of carboxymethyl cellulose, and accordingly, easily exerts an effect as a viscosity modifier. The high solubility can also increase the dispersibility of an active material and other components in the formation of slurry for an electrode. In this specification, cellulose and a cellulose derivative used as a binder of an electrode include salts thereof.

The water-soluble polymers stabilize viscosity by being dissolved in water and allow stable dispersion of the active material and another material combined as a binder, such as styrene-butadiene rubber, in an aqueous solution. Furthermore, a water-soluble polymer is expected to be easily and stably adsorbed to an active material surface because it has a functional group. Many cellulose derivatives such as carboxymethyl cellulose have functional groups such as a hydroxyl group and a carboxyl group. Because of functional groups, polymers are expected to interact with each other and cover an active material surface in a large area.

In the case where the binder covering or being in contact with the active material surface forms a film, the film is expected to serve as a passivation film to suppress the decomposition of the electrolyte solution. Here, the passivation film refers to a film without electronic conductivity or a film with extremely low electric conductivity, and can suppress the decomposition of an electrolyte solution at a potential at which a battery reaction occurs in the case where the passivation film is formed on the active material surface, for example. It is preferred that the passivation film can conduct lithium ions while suppressing electric conduction.

### <Positive Electrode Current Collector>

The positive electrode current collector can be formed using a material that has high conductivity, such as a metal like stainless steel, gold, platinum, aluminum, and titanium, or an alloy thereof. It is preferred that a material used for the positive electrode current collector does not dissolve at the potential of the positive electrode. Alternatively, it is possible to use an aluminum alloy to which an element that improves heat resistance, such as silicon, titanium, neodymium, scandium, or molybdenum, is added. Still alternatively, the positive electrode current collector may be formed using a metal element that forms silicide by reacting with silicon. Examples of the metal element that forms silicide by reacting with silicon include zirconium, titanium, hafnium, vanadium, niobium, tantalum, chromium, molybdenum, tungsten, cobalt, and nickel. The current collector can have any of various shapes including a foil-like shape, a plate-like shape (sheet-like shape), a net-like shape, a punching-metal shape, and an expanded-metal shape. The current collector preferably has a thickness of greater than or equal to 5 μm and less than or equal to 30 μm.

### [Negative Electrode]

The negative electrode includes a negative electrode active material layer and a negative electrode current collector. The negative electrode active material layer may contain a conductive additive and a binder.

### <Negative Electrode Active Material>

As a negative electrode active material, for example, an alloy-based material or a carbon-based material can be used.

For the negative electrode active material, an element that enables charge-discharge reactions by an alloying reaction and a dealloying reaction with lithium can be used. For example, a material containing at least one of silicon, tin, gallium, aluminum, germanium, lead, antimony, bismuth, silver, zinc, cadmium, indium, and the like can be used. Such elements have higher capacity than carbon. In particular, silicon has a high theoretical capacity of 4200 mAh/g. For this reason, silicon is preferably used as the negative electrode active material. Alternatively, a compound containing any of the above elements may be used. Examples of the compound include SiO, Mg₂Si, Mg₂Ge, SnO, SnO₂, Mg₂Sn, SnS₂, V₂Sn₃, FeSn₂, CoSn₂, Ni₃Sn₂, Cu₆Sn₅, Ag₃Sn, Ag₃Sb, Ni₂MnSb, CeSb₃, LaSn₃, La₃Co₂Sn₇, CoSb₃, InSb, and SbSn. Here, an element that enables charge-discharge reactions by an alloying reaction and a dealloying reaction with lithium and a compound containing the element, for example, may be referred to as an alloy-based material.

In this specification and the like, SiO refers, for example, to silicon monoxide. Note that SiO can alternatively be expressed as SiOₓ. Here, x preferably has an approximate value of 1. For example, x is preferably 0.2 or more and 1.5 or less, more preferably 0.3 or more and 1.2 or less.

As the carbon-based material, graphite, graphitizing carbon (soft carbon), non-graphitizing carbon (hard carbon), carbon nanotube, graphene, carbon black, and the like may be used.

Examples of graphite include artificial graphite and natural graphite. Examples of artificial graphite include meso-carbon microbeads (MCMB), coke-based artificial graphite, and pitch-based artificial graphite. As artificial graphite, spherical graphite having a spherical shape can be used. For example, MCMB is preferably used because it may have a spherical shape. Moreover, MCMB may preferably be used because it is relatively easy to have a small surface area. Examples of natural graphite include flake graphite and spherical natural graphite.

Graphite has a low potential substantially equal to that of a lithium metal (higher than or equal to 0.05 V and lower than or equal to 0.3 V vs. Li/Li⁺) when lithium ions are intercalated into graphite (while a lithium-graphite intercalayer compound is formed). For this reason, a lithium-ion secondary battery can have a high operating voltage. In addition, graphite is preferred because of its advantages such as a relatively high capacity per unit volume, relatively small volume expansion, low cost, and higher level of safety than that of a lithium metal.

Alternatively, for the negative electrode active material, oxide such as titanium dioxide (TiO₂), lithium titanium oxide (Li₄Ti₅O₁₂), lithium-graphite intercalation compound (LiₓC₆), niobium pentoxide (Nb₂O₅), tungsten oxide (WO₂), or molybdenum oxide (MoO₂) can be used.

Still alternatively, for the negative electrode active material, Li₃₋ₓMₓN (M=Co, Ni, or Cu) with a Li₃N structure, which is a nitride containing lithium and a transition metal, can be used. For example, Li_{2.6}Co_{0.4}N₃ is preferable because of high charge and discharge capacity (900 mAh/g and 1890 mAh/cm³).

A nitride containing lithium and a transition metal is preferably used, in which case lithium ions are contained in the negative electrode active material and thus the negative electrode active material can be used in combination with a material for a positive electrode active material which does not contain lithium ions, such as V₂O₅ or Cr₃O₈. Note that in the case of using a material containing lithium ions as a positive electrode active material, the nitride containing lithium and a transition metal can be used for the negative electrode active material by extracting the lithium ions contained in the positive electrode active material in advance.

Alternatively, a material that causes a conversion reaction can be used for the negative electrode active material. For example, a transition metal oxide that does not form an alloy with lithium, such as cobalt oxide (CoO), nickel oxide (NiO), and iron oxide (FeO), may be used. Other examples of the material that causes a conversion reaction include oxides such as Fe₂O₃, CuO, Cu₂O, RuO₂, and Cr₂O₃, sulfides such as CoS_{0.89}, NiS, and CuS, nitrides such as Zn₃N₂, Cu₃N, and Ge₃N₄, phosphides such as NiP₂, FeP₂, and CoP₃, and fluorides such as FeF₃ and BiF₃.

For the conductive additive and the binder that can be included in the negative electrode active material layer, materials similar to those of the conductive additive and the binder that can be included in the positive electrode active material layer can be used.

### <Negative Electrode Current Collector>

For the negative electrode current collector, a material similar to that of the positive electrode current collector can be used. Note that a material which is not alloyed with carrier ions such as lithium is preferably used for the negative electrode current collector.

### [Electrolyte Solution]

The electrolyte solution contains a solvent and an electrolyte. As the solvent of the electrolyte solution, an aprotic organic solvent is preferably used. For example, one of ethylene carbonate (EC), propylene carbonate (PC), butylene carbonate, chloroethylene carbonate, vinylene carbonate, γ-butyrolactone, γ-valerolactone, dimethyl carbonate (DMC), diethyl carbonate (DEC), ethyl methyl carbonate (EMC), methyl formate, methyl acetate, ethyl acetate, methyl propionate, ethyl propionate, propyl propionate, methyl butyrate, 1,3-dioxane, 1,4-dioxane, dimethoxyethane (DME), dimethyl sulfoxide, diethyl ether, methyl diglyme, acetonitrile, benzonitrile, tetrahydrofuran, sulfolane, and sultone can be used, or two or more of these solvents can be used in an appropriate combination in an appropriate ratio.

The use of one or more kinds of ionic liquids (room temperature molten salts) which have non-flammability and non-volatility as a solvent of the electrolyte solution can prevent a power storage device from exploding or catching fire even when the power storage device internally shorts out or the internal temperature increases owing to overcharging or the like. An ionic liquid contains a cation and an anion, specifically, an organic cation and an anion. Examples of the organic cation used for the electrolyte solution include aliphatic onium cations such as a quaternary ammonium cation, a tertiary sulfonium cation, and a quaternary phosphonium cation, and aromatic cations such as an imidazolium cation and a pyridinium cation. Examples of the anion used for the electrolyte solution include a monovalent amide-based anion, a monovalent methide-based anion, a fluorosulfonate anion, a perfluoroalkylsulfonate anion, a tetrafluoroborate anion, a perfluoroalkylborate anion, a hexafluorophosphate anion, and a perfluoroalkylphosphate anion.

As an electrolyte dissolved in the above-described solvent, one of lithium salts such as LiPF₆, LiClO₄, LiAsF₆, LiBF₄, LiAlCl₄, LiSCN, LiBr, LiI, Li₂SO₄, Li₂B₁₀Cl₁₀, Li₂B₁₂Cl₁₂, LiCF₃SO₃, LiC₄F₉SO₃, LiC(CF₃SO₂)₃, LiC(C₂F₅SO₂)₃, LiN(CF₃SO₂)₂, LiN(C₄F₉SO₂) (CF₃SO₂), and LiN(C₂F₅SO₂)₂ can be used, or two or more of these lithium salts can be used in an appropriate combination in an appropriate ratio.

The electrolyte solution used for a power storage device is preferably highly purified and contains a small amount of dust particles and elements other than the constituent elements of the electrolyte solution (hereinafter, also simply referred to as impurities). Specifically, the weight ratio of impurities to the electrolyte solution is less than or equal to 1%, preferably less than or equal to 0.1%, further preferably less than or equal to 0.01%.

Furthermore, an additive agent such as vinylene carbonate, propane sultone (PS), tert-butylbenzene (TBB), fluoroethylene carbonate (FEC), lithium bis(oxalate)borate (LiBOB), or a dinitrile compound such as succinonitrile or adiponitrile may be added to the electrolyte solution. The concentration of the additive agent in the whole solvent is, for example, higher than or equal to 0.1 weight % and lower than or equal to 5 weight %.

Alternatively, a polymer gelled electrolyte obtained in such a manner that a polymer is swelled with an electrolyte solution may be used.

When a polymer gel electrolyte is used, safety against liquid leakage and the like is improved. Furthermore, a secondary battery can be thinner and more lightweight.

As a polymer that undergoes gelation, a silicone gel, an acrylic gel, an acrylonitrile gel, a polyethylene oxide-based gel, a polypropylene oxide-based gel, a fluorine-based polymer gel, or the like can be used. Examples of the polymer include a polymer having a polyalkylene oxide structure, such as polyethylene oxide (PEO); PVDF; polyacrylonitrile; and a copolymer containing any of them. For example, PVDF-HFP, which is a copolymer of PVDF and hexafluoropropylene (HFP) can be used. The formed polymer may be porous.

Instead of the electrolyte solution, a solid electrolyte including an inorganic material such as a sulfide-based inorganic material or an oxide-based inorganic material, or a solid electrolyte including a high-molecular material such as a PEO (polyethylene oxide)-based high-molecular material may alternatively be used. When the solid electrolyte is used, a separator and a spacer are not necessary. Furthermore, the battery can be entirely solidified; therefore, there is no possibility of liquid leakage and thus the safety of the battery is dramatically increased.

### Embodiment 4

In this embodiment, examples of a shape of a secondary battery containing the positive electrode active material 100 described in the above embodiment are described. For the materials used for the secondary battery described in this embodiment, the description of the above embodiment can be referred to.

### [Coin Secondary Battery]

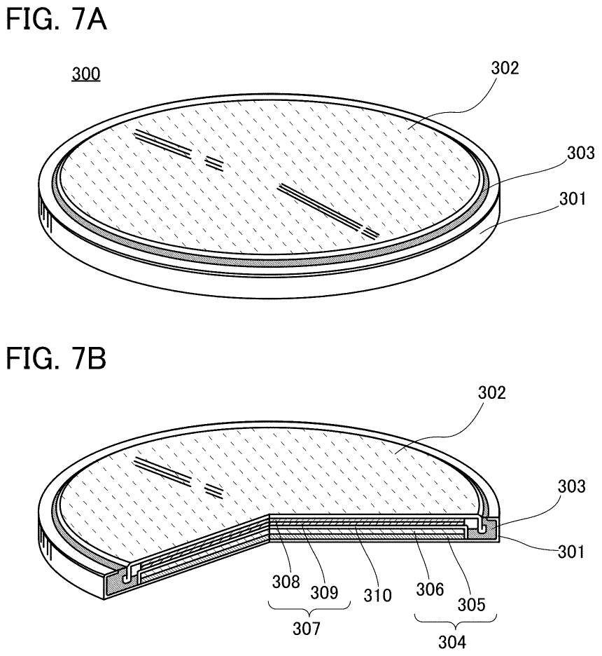

First, an example of a coin secondary battery is described. FIG. 7A is an external view of a coin (single-layer flat type) secondary battery, and FIG. 7B is a cross-sectional view thereof.

In a coin secondary battery 300, a positive electrode can 301 doubling as a positive electrode terminal and a negative electrode can 302 doubling as a negative electrode terminal are insulated from each other and sealed by a gasket 303 made of polypropylene or the like. A positive electrode 304 includes a positive electrode current collector 305 and a positive electrode active material layer 306 provided in contact with the positive electrode current collector 305. A negative electrode 307 includes a negative electrode current collector 308 and a negative electrode active material layer 309 provided in contact with the negative electrode current collector 308.

Note that only one surface of each of the positive electrode 304 and the negative electrode 307 used for the coin secondary battery 300 is provided with an active material layer.

For the positive electrode can 301 and the negative electrode can 302, a metal having corrosion resistance to an electrolyte solution, such as nickel, aluminum, or titanium, an alloy of such metals, or an alloy of such a metal and another metal (e.g., stainless steel) can be used. Alternatively, the positive electrode can 301 and the negative electrode can 302 are preferably covered with nickel, aluminum, or the like in order to prevent corrosion due to the electrolyte solution. The positive electrode can 301 and the negative electrode can 302 are electrically connected to the positive electrode 304 and the negative electrode 307, respectively.

The negative electrode 307, the positive electrode 304, and a separator 310 are immersed in the electrolyte solution. Then, as shown in FIG. 7B, the positive electrode 304, the separator 310, the negative electrode 307, and the negative electrode can 302 are stacked in this order with the positive electrode can 301 positioned at the bottom, and the positive electrode can 301 and the negative electrode can 302 are subjected to pressure bonding with the gasket 303 located therebetween. In such a manner, the coin secondary battery 300 is manufactured.

When the positive electrode active material particle described in the above embodiment is used in the positive electrode 304, the coin secondary battery 300 with little deterioration and high safety can be obtained.

### [Separator]

The secondary battery preferably includes a separator. As the separator, for example, fiber containing cellulose such as paper; nonwoven fabric; glass fiber; ceramics; synthetic fiber using nylon (polyamide), vinylon (polyvinyl alcohol-based fiber), polyester, acrylic, polyolefin, or polyurethane; or the like can be used. The separator is preferably formed to have an envelope-like shape to wrap one of the positive electrode and the negative electrode.

The separator may have a multilayer structure. For example, an organic material film such as polypropylene or polyethylene can be coated with a ceramic-based material, a fluorine-based material, a polyamide-based material, a mixture thereof, or the like. Examples of the ceramic-based material include aluminum oxide particles and silicon oxide particles. Examples of the fluorine-based material include PVDF and polytetrafluoroethylene. Examples of the polyamide-based material include nylon and aramid (meta-based aramid and para-based aramid).

Deterioration of the separator in charging and discharging at a high voltage can be suppressed and thus the reliability of the secondary battery can be improved because oxidation resistance is improved when the separator is coated with the ceramic-based material. In addition, when the separator is coated with the fluorine-based material, the separator is easily brought into close contact with an electrode, resulting in high output characteristics. When the separator is coated with the polyamide-based material, in particular, aramid, the safety of the secondary battery is improved because heat resistance is improved.

For example, both surfaces of a polypropylene film may be coated with a mixed material of aluminum oxide and aramid. Alternatively, a surface of the polypropylene film that is in contact with the positive electrode may be coated with the mixed material of aluminum oxide and aramid, and a surface of the polypropylene film that is in contact with the negative electrode may be coated with the fluorine-based material.

With the use of a separator having a multilayer structure, the capacity per volume of the secondary battery can be increased because the safety of the secondary battery can be maintained even when the total thickness of the separator is small.

### [Cylindrical Secondary Battery]

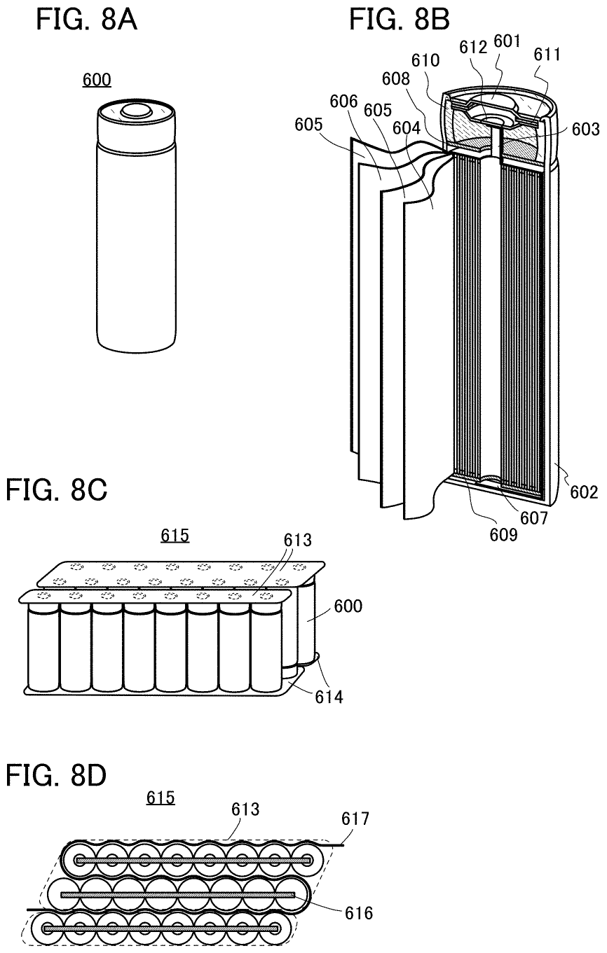

Examples of cylindrical secondary batteries are described with reference to FIG. 8A to FIG. 8D. As shown in FIG. 8A to FIG. 8B, the cylindrical secondary battery 600 includes a positive electrode cap (battery lid) 601 on a top surface and a battery can (outer can) 602 on a side surface and a bottom surface. The positive electrode cap and the battery can (outer can) 602 are insulated from each other by a gasket (insulating gasket) 610.

FIG. 8B is a schematic cross-sectional view of a cylindrical secondary battery. Inside the battery can 602 having a hollow cylindrical shape, a battery element in which a strip-like positive electrode 604 and a strip-like negative electrode 606 are wound with a separator 605 located therebetween is provided. The battery element is wound centering around a center pin, which is not shown. One end of the battery can 602 is close and the other end thereof is open. For the battery can 602, a metal having corrosion resistance to an electrolyte solution, such as nickel, aluminum, or titanium, an alloy of such a metal, or an alloy of such a metal and another metal (e.g., stainless steel) can be used. Alternatively, the battery can 602 is preferably covered with nickel, aluminum, or the like in order to prevent corrosion due to the electrolyte solution. Inside the battery can 602, the battery element in which the positive electrode, the negative electrode, and the separator are wound is provided between a pair of insulating plates 608 and 609 that face each other. Furthermore, a nonaqueous electrolyte solution (not shown) is injected inside the battery can 602 provided with the battery element. As the nonaqueous electrolyte, a nonaqueous electrolyte that is similar to that for the coin secondary battery can be used.

Since a positive electrode and a negative electrode that are used for a cylindrical storage battery are wound, active materials are preferably formed on both surfaces of a current collector. A positive electrode terminal (positive electrode current collecting lead) 603 is connected to the positive electrode 604, and a negative electrode terminal (negative electrode current collecting lead) 607 is connected to the negative electrode 606. Both the positive electrode terminal 603 and the negative electrode terminal 607 can be formed using a metal material such as aluminum. The positive electrode terminal 603 and the negative electrode terminal 607 are resistance-welded to a safety valve mechanism 612 and the bottom of the battery can 602, respectively. The safety valve mechanism 612 is electrically connected to the positive electrode cap 601 through a positive temperature coefficient (PTC) element 611. The safety valve mechanism 612 cuts off electrical connection between the positive electrode cap 601 and the positive electrode 604 when the internal pressure of the battery exceeds a predetermined threshold value. The PTC element 611, which serves as a thermally sensitive resistor whose resistance increases as temperature rises, limits the amount of current by increasing the resistance, in order to prevent abnormal heat generation. Barium titanate (BaTiO₃)-based semiconductor ceramics or the like can be used for the PTC element.

Alternatively, as shown in FIG. 8C, a plurality of secondary batteries 600 may be provided between a conductive plate 613 and a conductive plate 614 to form a module 615. The plurality of secondary batteries 600 may be connected in parallel, connected in series, or connected in series after being connected in parallel. With the module 615 including the plurality of secondary batteries 600, large electric power can be extracted.

FIG. 8D is atop view of the module 615. The conductive plate 613 is shown by a dotted line for clarity of the drawing. As shown in FIG. 8D, the module 615 may include a wiring 616 electrically connecting the plurality of secondary batteries 600 with each other. It is possible to provide the conductive plate over the wiring 616 to overlap with each other. In addition, a temperature control device 617 may be provided between the plurality of secondary batteries 600. The secondary batteries 600 can be cooled with the temperature control device 617 when overheated, whereas the secondary batteries 600 can be heated with the temperature control device 617 when cooled too much. Thus, the performance of the module 615 is less likely to be influenced by the outside temperature.

When the positive electrode active material 100 described in the above embodiment is used in the positive electrode 604, the cylindrical secondary battery 600 with little deterioration and high safety can be obtained.

### [Structural Example of Power Storage Device]

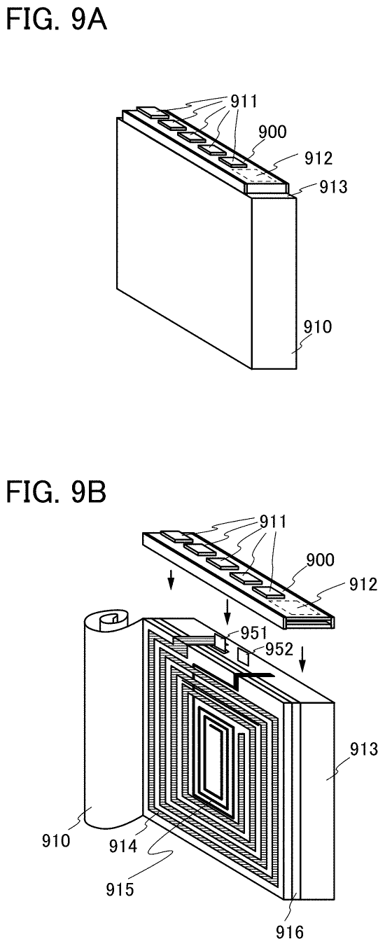

Other structural examples of power storage devices are described with reference to FIG. 9 to FIG. 13.

FIG. 9A and FIG. 9B are external views of a power storage device. The power storage device includes a circuit board 900 and a secondary battery 913. A label 910 is attached to the secondary battery 913. The power storage device further includes a terminal 951, a terminal 952, an antenna 914, and an antenna 915 as shown in FIG. 9B.

The circuit board 900 includes a terminal 911 and a circuit 912. The terminal 911 is connected to the terminal 951, the terminal 952, the antenna 914, the antenna 915, and the circuit 912. Note that a plurality of terminals 911 may be provided and each of the plurality of terminals 911 may serve as a control signal input terminal, a power supply terminal, and the like.

The circuit 912 may be provided on the rear surface of the circuit board 900. Note that the shapes of the antenna 914 and the antenna 915 are not limited to coil shapes, and may be linear shapes or plate shapes, for example. An antenna such as a planar antenna, an aperture antenna, a traveling-wave antenna, an EH antenna, a magnetic-field antenna, or a dielectric antenna may be used. Alternatively, the antenna 914 or the antenna 915 may be a flat-plate conductor. This flat-plate conductor can serve as one of conductors for electric field coupling. That is, the antenna 914 or the antenna 915 may serve as one of two conductors of a capacitor. Thus, electric power can be transmitted and received not only by an electromagnetic field or a magnetic field but also by an electric field.

The line width of the antenna 914 is preferably larger than the line width of the antenna 915. This makes it possible to increase the amount of power received by the antenna 914.

The power storage device includes a layer 916 between the secondary battery 913, and the antenna 914 and the antenna 915. The layer 916 has a function of, for example, blocking an electromagnetic field from the secondary battery 913. As the layer 916, for example, a magnetic body can be used.

Note that the structure of the power storage device is not limited to that shown in FIG. 9.

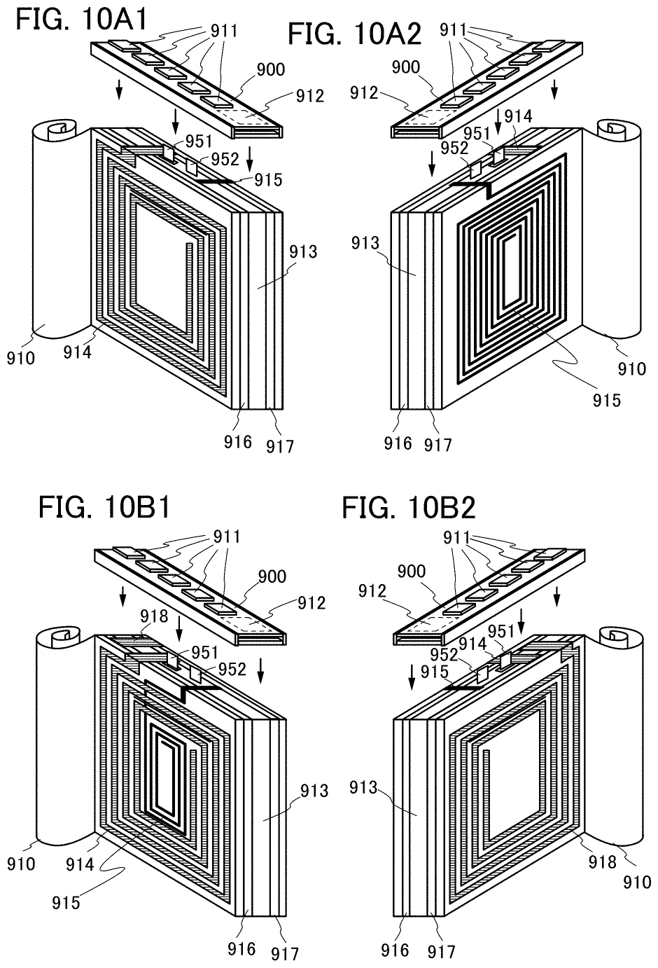

For example, as shown in FIG. 10A1 and FIG. 10A2, an antenna may be provided for each of a pair of opposing surfaces of the secondary battery 913 shown in FIG. 9A and FIG. 9B. FIG. 10A1 is an external view showing one side of the above pair of surfaces, and FIG. 10A2 is an external view showing the other side of the above pair of surfaces. For portions similar to those shown in FIG. 9A and FIG. 9B, a description of the storage device shown in FIG. 9A and FIG. 9B can be referred to as appropriate.

As shown in FIG. 10A1, the antenna 914 is provided on one of the pair of surfaces of the secondary battery 913 with the layer 916 located therebetween, and as shown in FIG. 10A2, an antenna 915 is provided on the other of the pair of surfaces of the secondary battery 913 with a layer 917 located therebetween. The layer 917 has a function of, for example, blocking an electromagnetic field from the secondary battery 913. As the layer 917, for example, a magnetic body can be used.

With the above structure, both the antenna 914 and the antenna 915 can be increased in size.

Alternatively, as shown in FIG. 10B1 and FIG. 10B2, a pair of opposing surfaces of the secondary battery 913 in FIG. 9A and FIG. 9B may be provided with different types of antennas. FIG. 10B1 is an external view seen from the direction of one side of the above-described pair of surfaces, and FIG. 10B2 is an external view seen from the direction of the other side of the above-described pair of surfaces. For portions similar to those shown in FIG. 9A and FIG. 9B, a description of the storage device shown in FIG. 9A and FIG. 9B can be referred to as appropriate.

As shown in FIG. 10B1, the antenna 914 and the antenna 915 are provided on one of the opposing surfaces of the secondary battery 913 with the layer 916 interposed therebetween, and as shown in FIG. 10B2, an antenna 918 is provided on the other of the opposing surfaces of the secondary battery 913 with the layer 917 interposed therebetween. The antenna 918 has a function of communicating data with an external device, for example. An antenna with a shape that can be applied to the antenna 914 and the antenna 915, for example, can be used as the antenna 918. As a system for communication using the antenna 918 between the power storage device and another device, a response method that can be used between the power storage device and another device, such as NFC, can be employed.

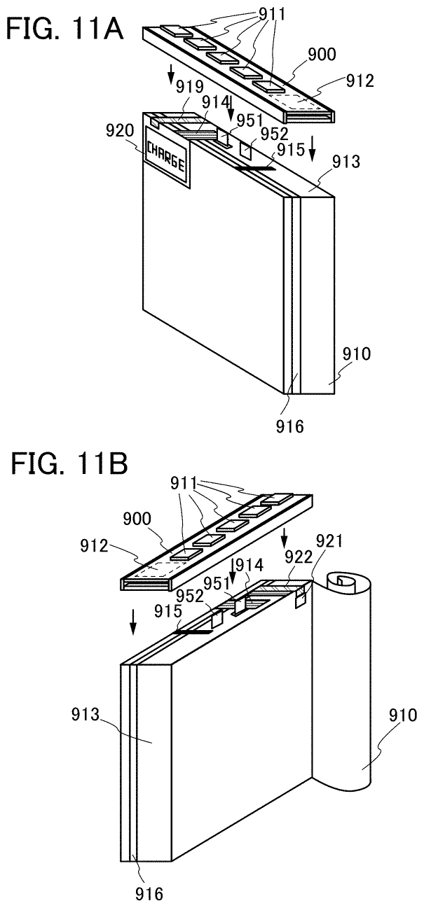

Alternatively, as shown in FIG. 11A, the secondary battery 913 shown in FIG. 9A and FIG. 9B may be provided with a display device 920. The display device 920 is electrically connected to the terminal 911 through a terminal 919. Note that the label 910 is not necessarily provided in a portion where the display device 920 is provided. For portions similar to those shown in FIG. 9A and FIG. 9B, a description of the storage device shown in FIG. 9A and FIG. 9B can be referred to as appropriate.

The display device 920 may display, for example, an image showing whether charging is being carried out, an image showing the amount of stored power, or the like. As the display device 920, electronic paper, a liquid crystal display device, an electroluminescent (EL) display device, or the like can be used. For example, the use of electronic paper can reduce power consumption of the display device 920.

Alternatively, as shown in FIG. 11B, the secondary battery 913 shown in FIG. 9A and FIG. 9B may be provided with a sensor 921. The sensor 921 is electrically connected to the terminal 911 through a terminal 922. For portions similar to those shown in FIG. 9A and FIG. 9B, a description of the storage device shown in FIG. 9A and FIG. 9B can be referred to as appropriate.

The sensor 921 has a function of measuring, for example, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays. With the sensor 921, for example, data on an environment (e.g., temperature) where the storage device is placed can be determined and stored in a memory inside the circuit 912.

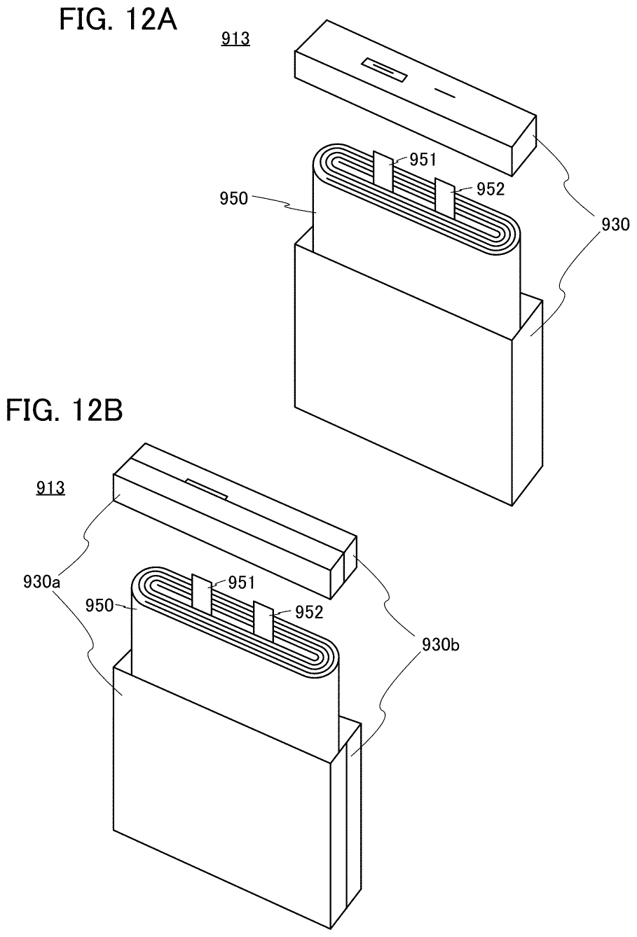

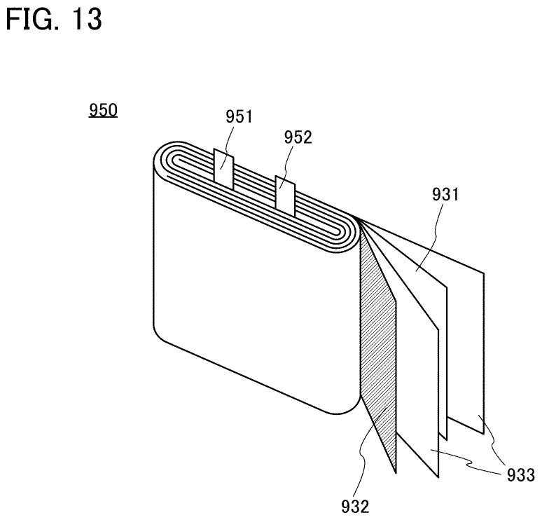

Furthermore, structure examples of the secondary battery 913 are described using FIG. 12 and FIG. 13.

The secondary battery 913 shown in FIG. 12A includes a wound body 950 provided with the terminal 951 and the terminal 952 inside a housing 930. The wound body 950 is soaked in an electrolyte solution inside the housing 930. The terminal 952 is in contact with the housing 930. An insulator or the like inhibits the contact between the terminal 951 and the housing 930. Note that in FIG. 12A, the housing 930 divided into two pieces is shown for convenience; however, in the actual structure, the wound body 950 is covered with the housing 930 and the terminal 951 and the terminal 952 extend to the outside of the housing 930. For the housing 930, a metal material (e.g., aluminum) or a resin material can be used.

Note that as shown in FIG. 12B, the housing 930 shown in FIG. 12A may be formed using a plurality of materials. For example, in the secondary battery 913 shown in FIG. 12B, a housing 930a and a housing 930b are bonded to each other, and the wound body 950 is provided in a region surrounded by the housing 930a and the housing 930b.

For the housing 930a, an insulating material such as an organic resin can be used. In particular, when a material such as an organic resin is used for the side on which an antenna is formed, blocking of an electric field from the secondary battery 913 can be inhibited. When an electric field is not significantly blocked by the housing 930a, an antenna such as the antenna 914 and the antenna 915 may be provided inside the housing 930a. For the housing 930b, a metal material can be used, for example.

FIG. 13 shows the structure of the wound body 950. The wound body 950 includes a negative electrode 931, a positive electrode 932, and separators 933. The wound body 950 is obtained by winding a sheet of a stack in which the negative electrode 931 overlaps with the positive electrode 932 with the separator 933 provided therebetween. Note that a plurality of stacks each including the negative electrode 931, the positive electrode 932, and the separator 933 may be stacked.

The negative electrode 931 is connected to the terminal 911 shown in FIG. 9 through one of the terminal 951 and the terminal 952. The positive electrode 932 is connected to the terminal 911 shown in FIG. 7 through the other of the terminal 951 and the terminal 952.

When the positive electrode active material particle described in the above embodiment is used in the positive electrode 932, the secondary battery 913 with little deterioration and high safety can be obtained.

### [Laminated Secondary Battery]

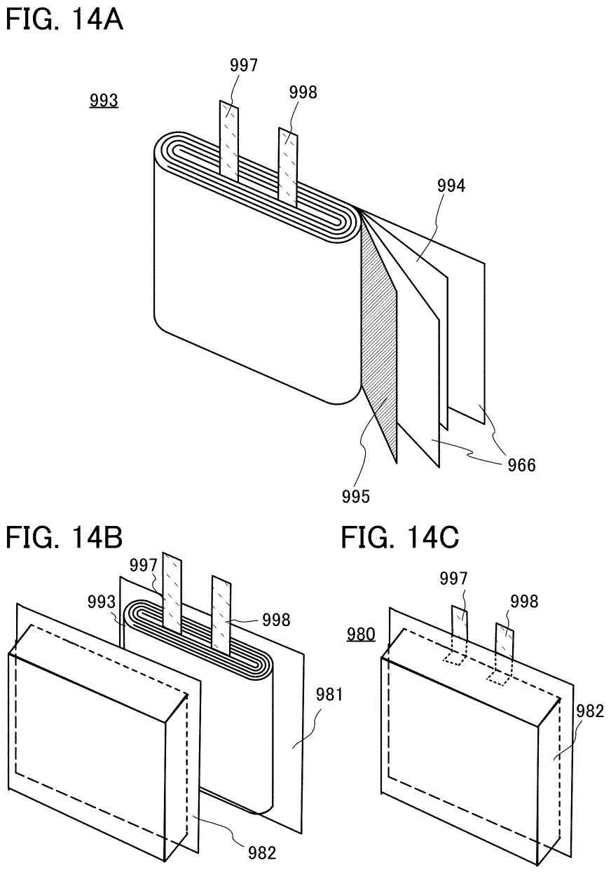

Next, examples of laminated secondary batteries are described with reference to FIG. 14 to FIG. 19. When the laminated secondary battery has flexibility and is used in an electronic device at least part of which is flexible, the secondary battery can be bent as the electronic device is bent.

A laminated secondary battery 980 is described using FIG. 14. The laminated secondary battery 980 includes a wound body 993 shown in FIG. 14A. The wound body 993 includes a negative electrode 994, a positive electrode 995, and separators 996. The wound body 993 is, like the wound body 950 shown in FIG. 13, obtained by winding a sheet of a stack in which the negative electrode 994 overlaps with the positive electrode 995 with the separator 996 provided therebetween.

Note that the number of stacks each including the negative electrode 994, the positive electrode 995, and the separator 996 may be determined as appropriate depending on required capacity and element volume. The negative electrode 994 is connected to a negative electrode current collector (not shown) through one of a lead electrode 997 and a lead electrode 998. The positive electrode 995 is connected to a positive electrode current collector (not shown) through the other of the lead electrode 997 and the lead electrode 998.

As shown in FIG. 14B, the wound body 993 is packed in a space formed by bonding a film 981 and a film 982 having a depressed portion that serve as exterior bodies by thermocompression bonding or the like, whereby the secondary battery 980 shown in FIG. 14C can be fabricated. The wound body 993 includes the lead electrode 997 and the lead electrode 998, and is soaked in an electrolyte solution inside a space surrounded by the film 981 and the film 982 having a depressed portion.

For the film 981 and the film 982 having a depressed portion, a metal material such as aluminum or a resin material can be used, for example. With the use of a resin material for the film 981 and the film 982 having a depressed portion, the film 981 and the film 982 having a depressed portion can be changed in their forms when external force is applied; thus, a flexible storage battery can be formed.

Although FIG. 14B and FIG. 14C show an example where a space is formed by two films, the wound body 993 may be placed in a space formed by bending one film.

When the positive electrode active material particle described in the above embodiment is used in the positive electrode 995, the secondary battery 980 with little deterioration and high safety can be obtained.

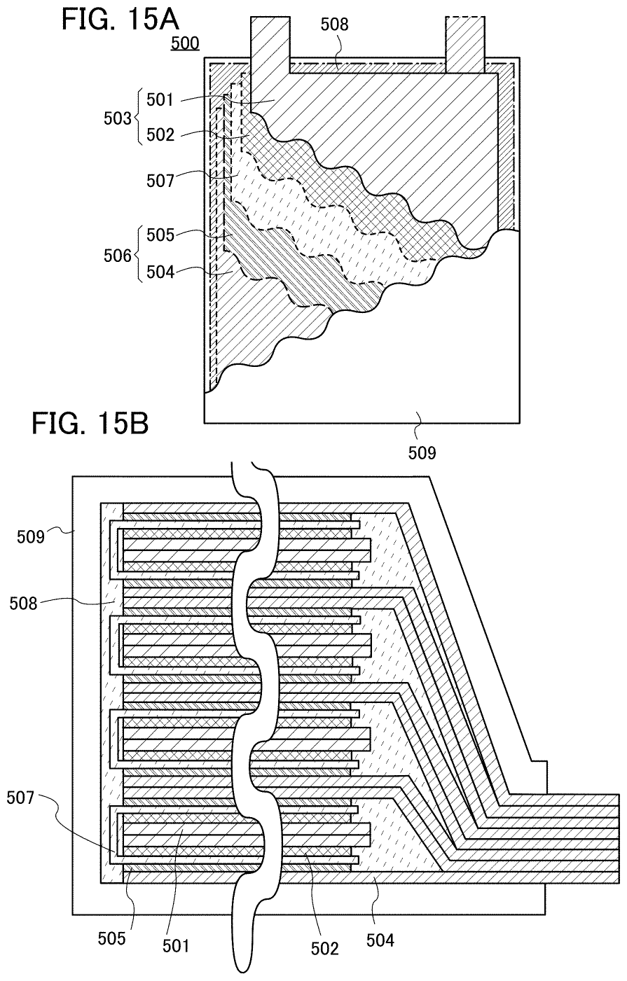

In addition, FIG. 14 shows an example in which the secondary battery 980 includes a wound body in a space formed by films serving as an exterior body; however, as shown in FIG. 15, for example, a secondary battery may include a plurality of strip-shaped positive electrodes, separators, and negative electrodes in a space formed by films serving as an exterior body.

A laminated secondary battery 500 shown in FIG. 15A includes a positive electrode 503 including a positive electrode current collector 501 and a positive electrode active material layer 502, a negative electrode 506 including a negative electrode current collector 504 and a negative electrode active material layer 505, a separator 507, an electrolyte solution 508, and an exterior body 509. The separator 507 is provided between the positive electrode 503 and the negative electrode 506 in the exterior body 509. The exterior body 509 is filled with the electrolyte solution 508. The electrolyte solution described in Embodiment 2 can be used as the electrolyte solution 508.

In the laminated secondary battery 500 shown in FIG. 15A, the positive electrode current collector 501 and the negative electrode current collector 504 also serve as terminals for electrical contact with the outside. For this reason, the positive electrode current collector 501 and the negative electrode current collector 504 may be arranged so that part of the positive electrode current collector 501 and part of the negative electrode current collector 504 are exposed to the outside of the exterior body 509. Alternatively, a lead electrode and the positive electrode current collector 501 or the negative electrode current collector 504 may be bonded to each other by ultrasonic welding, and instead of the positive electrode current collector 501 and the negative electrode current collector 504, the lead electrode may be exposed to the outside of the exterior body 509.

As the exterior body 509 of the laminated secondary battery 500, for example, a laminate film having a three-layer structure can be employed in which a highly flexible metal thin film of aluminum, stainless steel, copper, nickel, or the like is provided over a film formed of a material such as polyethylene, polypropylene, polycarbonate, ionomer, or polyamide, and an insulating synthetic resin film of a polyamide-based resin, a polyester-based resin, or the like is provided over the metal thin film as the outer surface of the exterior body.

FIG. 15B shows an example of a cross-sectional structure of the laminated secondary battery 500. Although FIG. 15A shows an example in which the laminated secondary battery 500 is composed of two current collectors for simplicity, the laminated secondary battery 500 is actually composed of a plurality of electrode layers.

In FIG. 15B, the number of electrode layers is 16, for example. The laminated secondary battery 500 has flexibility even though including 16 electrode layers. FIG. 15B shows a structure including 8 layers of negative electrode current collectors 504 and 8 layers of positive electrode current collectors 501, i.e., 16 layers in total. Note that FIG. 15B shows a cross section of the negative electrode extraction portion, and the 8 layers of the negative electrode current collectors 504 are bonded to each other by ultrasonic welding. It is needless to say that the number of electrode layers is not limited to 16, and may be more than 16 or less than 16. With a large number of electrode layers, the secondary battery can have high capacity. In contrast, with a small number of electrode layers, the secondary battery can have small thickness and high flexibility.

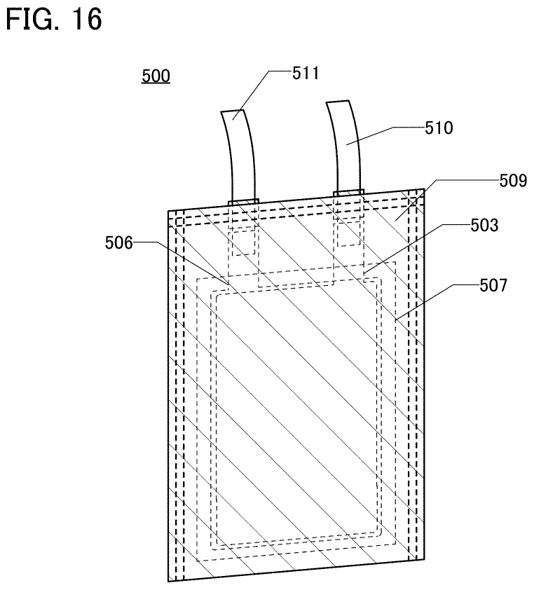

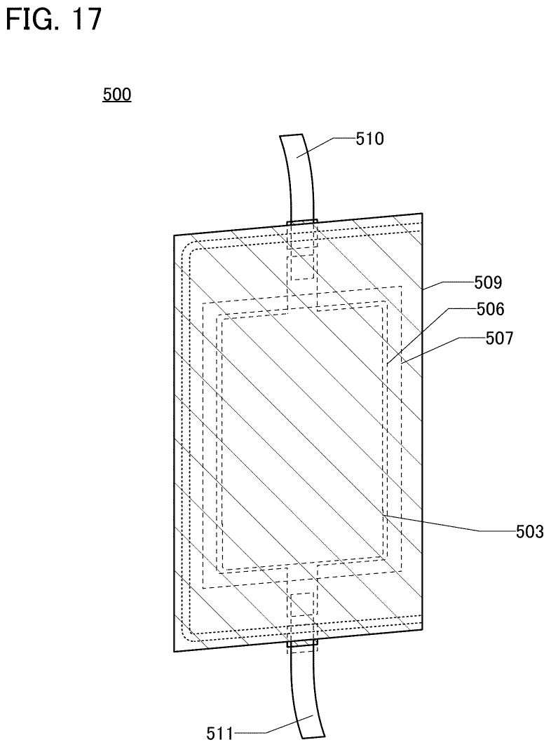

FIG. 16 and FIG. 17 show examples of the external views of the laminated secondary battery 500. In FIG. 16 and FIG. 17, the laminated secondary battery 500 includes the positive electrode 503, the negative electrode 506, the separator 507, the exterior body 509, a positive electrode lead electrode 510, and a negative electrode lead electrode 511.

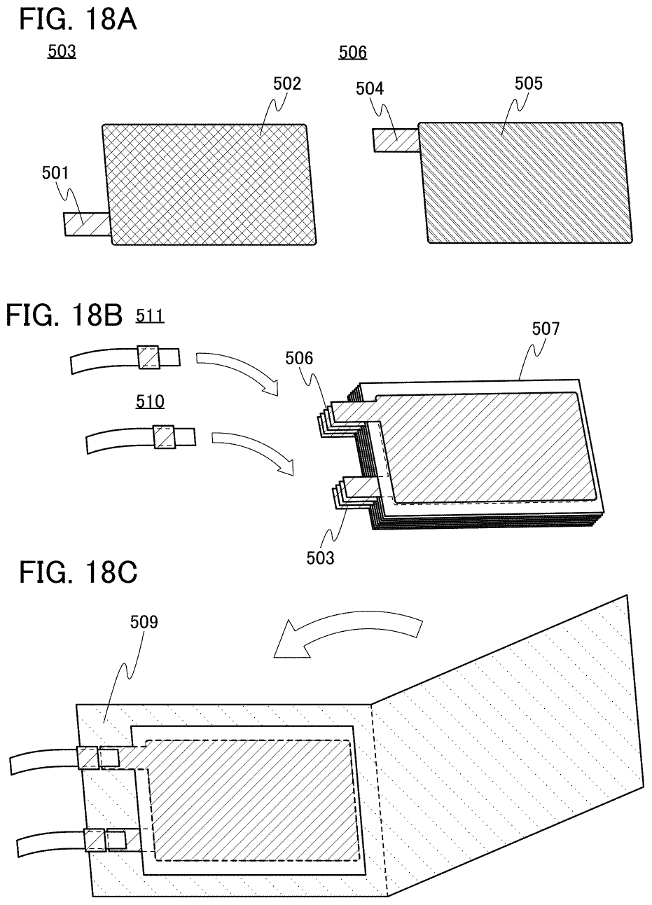

FIG. 18A shows external views of the positive electrode 503 and the negative electrode 506. The positive electrode 503 includes the positive electrode current collector 501, and the positive electrode active material layer 502 is formed on a surface of the positive electrode current collector 501. The positive electrode 503 also includes a region where the positive electrode current collector 501 is partly exposed (hereinafter, referred to as a tab region). The negative electrode 506 includes the negative electrode current collector 504, and the negative electrode active material layer 505 is formed on a surface of the negative electrode current collector 504. The negative electrode 506 also includes a region where the negative electrode current collector 504 is partly exposed, that is, a tab region. The areas and the shapes of the tab regions included in the positive electrode and the negative electrode are not limited to those shown in FIG. 18A.

### [Manufacturing Method of Laminated Secondary Battery]

Here, an example of a method of manufacturing the laminated secondary battery whose external view is shown in FIG. 16 is described with reference to FIG. 18B and FIG. 18C.

First, the negative electrode 506, the separator 507, and the positive electrode 503 are stacked. FIG. 18B shows a stack including the negative electrode 506, the separator 507, and the positive electrode 503. Here, an example in which 5 negative electrodes and 4 positive electrodes are used is shown. Next, the tab regions of the positive electrodes 503 are bonded to each other, and the tab region of the positive electrode on the outermost surface and the positive electrode lead electrode 510 are bonded to each other. The bonding can be performed by ultrasonic welding, for example. In a similar manner, the tab regions of the negative electrodes 506 are bonded to each other, and the tab region of the negative electrode on the outermost surface and the negative electrode lead electrode 511 are bonded to each other.

After that, the negative electrode 506, the separator 507, and the positive electrode 503 are placed over the exterior body 509.

Subsequently, the exterior body 509 is folded along a dashed line as shown in FIG. 18C. Then, the outer edges of the exterior body 509 are bonded to each other. The bonding can be performed by thermocompression, for example. At this time, an unbonded region (hereinafter referred to as an inlet) is provided for part (or one side) of the exterior body 509 so that the electrolyte solution 508 can be introduced later.

Next, the electrolyte solution 508 is introduced into the inside of the exterior body 509 from the inlet provided for the exterior body 509. The electrolyte solution 508 is preferably introduced in a reduced pressure atmosphere or in an inert gas atmosphere. Lastly, the inlet is sealed by bonding. In this manner, the secondary battery 500 that is a laminated secondary battery can be manufactured.

When the positive electrode active material particle described in the above embodiment is used in the positive electrode 503, the secondary battery 500 with little deterioration and high safety can be obtained.

### [Bendable Secondary Battery]

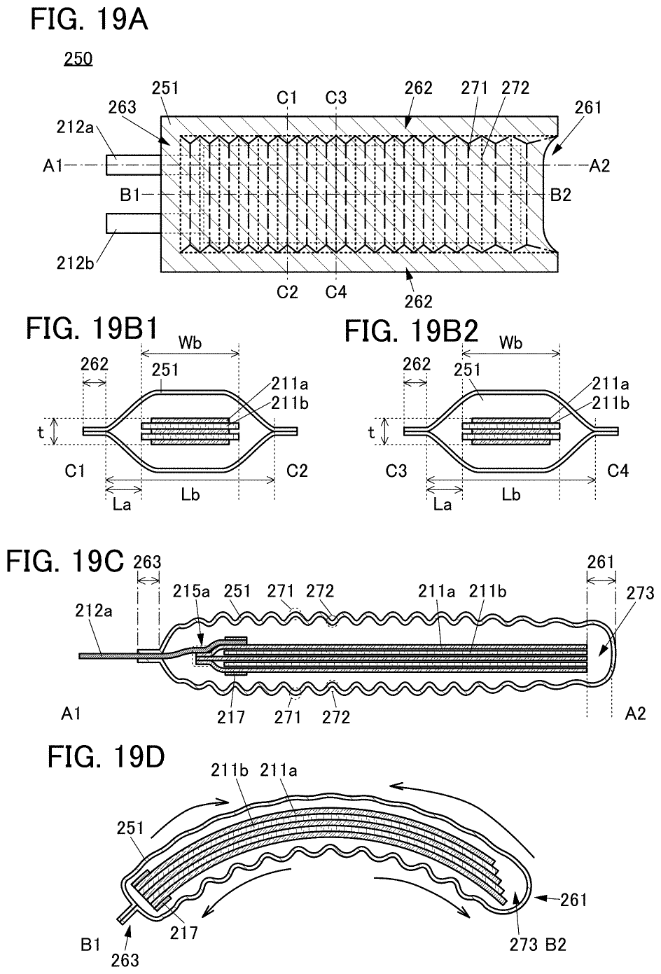

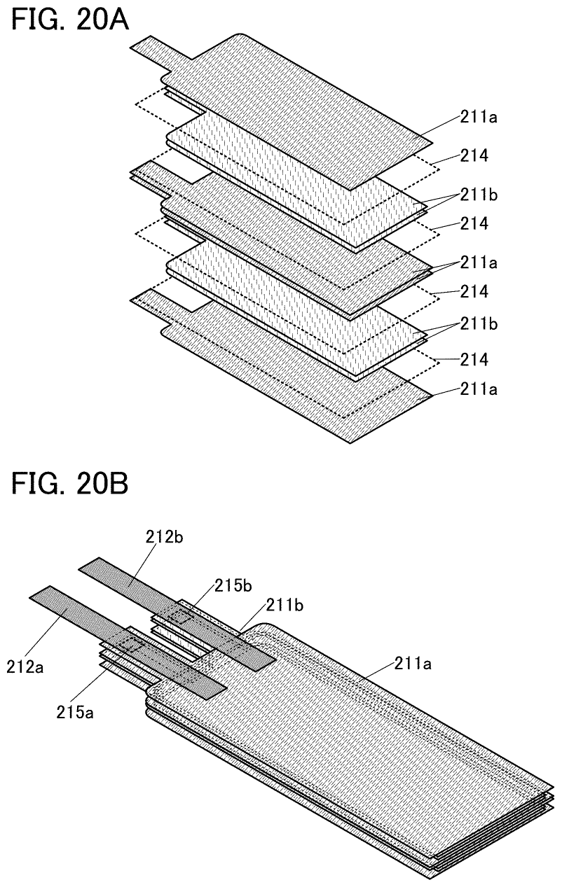

Next, examples of bendable secondary batteries are described with reference to FIG. 19 and FIG. 20.

FIG. 19A is a schematic top view of a bendable secondary battery 250. FIG. 19B1, FIG. 19B2, and FIG. 19C are schematic cross-sectional views taken along the dotted line C1-C2, the dotted line C3-C4, and the dotted line A1-A2, respectively, in FIG. 19A. The battery 250 includes an exterior body 251 and a positive electrode 211a, and a negative electrode 211b held in the exterior body 251. A lead 212a electrically connected to the positive electrode 211a and a lead 212b electrically connected to the negative electrode 211b are extended to the outside of the exterior body 251. In addition to the positive electrode 211a and the negative electrode 211b, an electrolyte solution (not shown) is enclosed in a region surrounded by the exterior body 251.

The positive electrode 211a and the negative electrode 211b included in the secondary battery 250 are described with reference to FIG. 20. FIG. 20A is a perspective view showing the stacking order of the positive electrode 211a, the negative electrode 211b, and a separator 214. FIG. 20B is a perspective view showing the lead 212a and the lead 212b in addition to the positive electrode 211a and the negative electrode 211b.

As shown in FIG. 20A, the secondary battery 250 includes a plurality of strip-shaped positive electrodes 211a, a plurality of strip-shaped negative electrodes 211b, and a plurality of separators 214. The positive electrode 211a and the negative electrode 211b each include a projected tab portion and a portion other than the tab. A positive electrode active material layer is formed on one surface of the positive electrode 211a other than the tab, and a negative electrode active material layer is formed on one surface of the negative electrode 211b other than the tab.

The positive electrodes 211a and the negative electrodes 211b are stacked so that surfaces of the positive electrodes 211a on each of which the positive electrode active material layer is not formed are in contact with each other and that surfaces of the negative electrodes 211b on each of which the negative electrode active material is not formed are in contact with each other.

Furthermore, the separator 214 is provided between the surface of the positive electrode 211a on which the positive electrode active material is formed and the surface of the negative electrode 211b on which the negative electrode active material is formed. In FIG. 20, the separator 214 is shown by a dotted line for easy viewing.

In addition, as shown in FIG. 20B, the plurality of positive electrodes 211a are electrically connected to the lead 212a in a bonding portion 215a. The plurality of negative electrodes 211b are electrically connected to the lead 212b in a bonding portion 215b.

Next, the exterior body 251 is described with reference to FIG. 19B1, FIG. 19B2, FIG. 19C, and FIG. 19D.

The exterior body 251 has a film-like shape and is folded in half so as to sandwich the positive electrodes 211a and the negative electrodes 211b. The exterior body 251 includes a folded portion 261, a pair of seal portions 262, and a seal portion 263. The pair of seal portions 262 is provided with the positive electrodes 211a and the negative electrodes 211b positioned therebetween and thus can also be referred to as side seals. The seal portion 263 includes portions overlapping with the lead 212a and the lead 212b and can also be referred to as a top seal.

Part of the exterior body 251 that overlaps with the positive electrodes 211a and the negative electrodes 211b preferably has a wave shape in which crest lines 271 and trough lines 272 are alternately arranged. The seal portions 262 and the seal portion 263 of the exterior body 251 are preferably flat.

FIG. 19B1 is a cross section cut along a portion overlapping with the crest line 271, and FIG. 19B2 is a cross section cut along a portion overlapping with the trough line 272. FIG. 19B1 and FIG. 19B2 correspond to cross sections of the battery 250, the positive electrodes 211a, and the negative electrodes 211b in the width direction.

Here, the distance between end portions of the positive electrode 211a and the negative electrode 211b in the width direction and the seal portion 262, that is, the distance between the end portions of the positive electrode 211a and the negative electrode 211b and the seal portion 262 is referred to as a distance La. When the battery 250 changes in shape, for example, is bent, the positive electrode 211a and the negative electrode 211b change in shape such that the positions thereof are shifted from each other in the length direction as described later. At the time, if the distance La is too short, the exterior body 251 and the positive electrode 211a and the negative electrode 211b are rubbed hard against each other, so that the exterior body 251 is damaged in some cases. In particular, when a metal film of the exterior body 251 is exposed, the metal film might be corroded by the electrolyte solution. Therefore, the distance La is preferably set as long as possible. However, if the distance La is too long, the volume of the battery 250 is increased.

The distance La between the positive and negative electrodes 211a and 211b and the seal portion 262 is preferably increased as the total thickness of the stacked positive electrodes 211a and negative electrodes 211b is increased.

More specifically, when the total thickness of the stacked positive electrodes 211a and negative electrodes 211b is referred to as a thickness t, the distance La is preferably 0.8 times or more and 3.0 times or less, further preferably 0.9 times or more and 2.5 times or less, still further preferably 1.0 times or more and 2.0 times or less as large as the thickness t. When the distance La is in the above range, a compact battery highly reliable for bending can be obtained.

Furthermore, when the distance between the pair of seal portions 262 is referred to as a distance Lb, it is preferred that the distance Lb be sufficiently longer than the widths of the positive electrode 211a and the negative electrode 211b (here, a width Wb of the negative electrode 211b). In this case, even when the positive electrode 211a and the negative electrode 211b come into contact with the exterior body 251 by change in the shape of the battery 250 such as repeated bending, the position of part of the positive electrode 211a and the negative electrode 211b can be shifted in the width direction; thus, the positive and negative electrodes 211a and 211b and the exterior body 251 can be effectively prevented from being rubbed against each other.

For example, the difference between the distance La between the pair of seal portions 262 and the width Wb of the negative electrode 211b is preferably 1.6 times or more and 6.0 times or less, further preferably 1.8 times or more and 5.0 times or less, still further preferably 2.0 times or more and 4.0 times or less as large as the total thickness t of the positive electrode 211a and the negative electrode 211b.

In other words, the distance Lb, the width Wb, and the thickness t preferably satisfy the relationship of Formula 7 below.

$$[ Formula 7 ]$$

$$\frac{Lb - Wb}{2 t} \geq a \qquad ( Formula 7 )$$

Here, a satisfies 0.8 or more and 3.0 or less, preferably 0.9 or more and 2.5 or less, further preferably 1.0 or more and 2.0 or less.

FIG. 19C shows a cross section including the lead 212a and corresponds to a cross section of the battery 250, the positive electrode 211a, and the negative electrode 211b in the length direction. As shown in FIG. 19C, in the folded portion 261, a space 273 is preferably included between the end portions of the positive electrode 211a and the negative electrode 211b in the length direction and the exterior body 251.

FIG. 19D is a schematic cross-sectional view of the battery 250 in a state of being bent. FIG. 19D corresponds to a cross section along the cutting line B1-B2 in FIG. 19A.

When the battery 250 is bent, part of the exterior body 251 positioned on the outer side in bending is stretched and the other part positioned on the inner side changes its shape as it is squashed. More specifically, the part of the exterior body 251 positioned on the outer side in bending changes its shape such that the wave amplitude becomes smaller and the length of the wave period becomes larger. In contrast, the part of the exterior body 251 positioned on the inner side changes its shape such that the wave amplitude becomes larger and the length of the wave period becomes smaller. When the exterior body 251 changes its shape in this manner, stress applied to the exterior body 251 with bending is relieved, so that a material itself of the exterior body 251 does not need to be stretched or squashed. As a result, the battery 250 can be bent with weak force without damage to the exterior body 251.

Furthermore, as shown in FIG. 19D, when the battery 250 is bent, the positions of the positive electrode 211a and the negative electrode 211b are shifted relatively. At this time, ends of the stacked positive electrodes 211a and negative electrodes 211b on the seal portion 263 side are fixed by a fixing member 217. Thus, the plurality of positive electrodes 211a and the plurality of negative electrodes 211b are more shifted at a position closer to the folded portion 261. Therefore, stress applied to the positive electrode 211a and the negative electrode 211b is relieved, and the positive electrode 211a and the negative electrode 211b themselves do not need to be stretched or squashed. As a result, the battery 250 can be bent without damage to the positive electrode 211a and the negative electrode 211b.

Furthermore, the space 273 is provided between the positive electrode 211a and the negative electrode 211b and the exterior body 251, whereby the positive electrode 211a and the negative electrode 211b located on an inner side can be shifted relatively without being in contact with the exterior body 251 when the battery 250 is bent.

In the battery 250 shown in FIG. 19 and FIG. 20, the exterior body, the positive electrode 211a, and the negative electrode 211b are less likely to be damaged and the battery characteristics are less likely to deteriorate even when the battery 250 is repeatedly bent and unbent. When the positive electrode active material particle described in the above embodiment is used for the positive electrode 211a included in the battery 250, a battery with little deterioration and high safety can be obtained.

### Embodiment 5

In this embodiment, examples of electronic devices each including the secondary battery of one embodiment of the present invention are described.

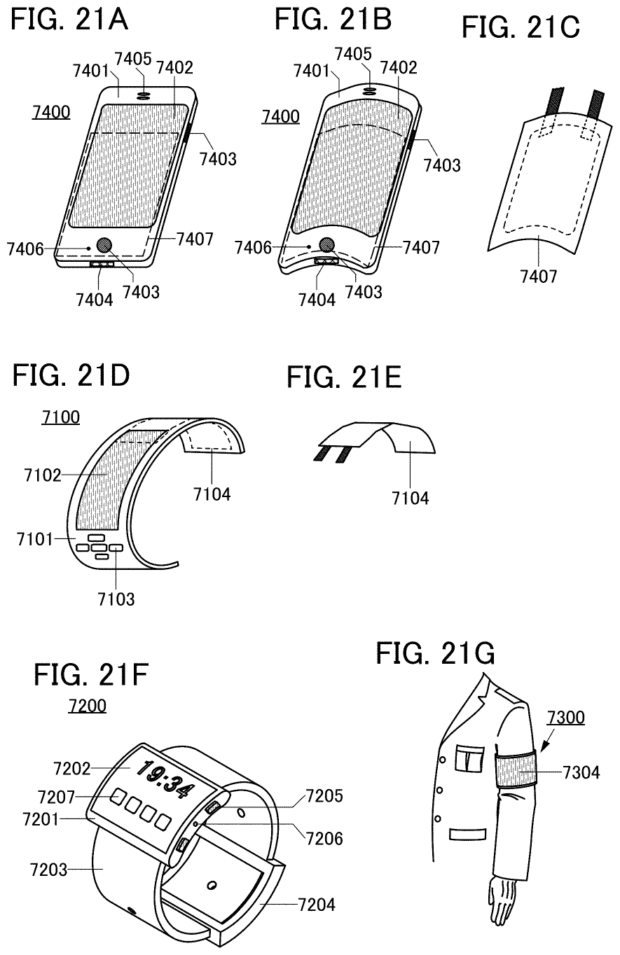

First, FIG. 21 shows examples of electronic devices including the bendable secondary battery described in Embodiment 3. Examples of electronic devices each including a bendable secondary battery include television sets (also referred to as televisions or television receivers), monitors of computers or the like, digital cameras, digital video cameras, digital photo frames, mobile phones (also referred to as cellular phones or mobile phone devices), portable game machines, portable information terminals, audio reproducing devices, and large game machines such as pachinko machines.

In addition, a flexible secondary battery can be incorporated along a curved inside/outside wall surface of a house or a building or a curved interior/exterior surface of an automobile.

FIG. 21A shows an example of a mobile phone. A mobile phone 7400 is provided with a display portion 7402 incorporated in a housing 7401, an operation button 7403, an external connection port 7404, a speaker 7405, a microphone 7406, and the like. Note that the mobile phone 7400 includes a secondary battery 7407.

FIG. 21B shows the bent mobile phone 7400. When the whole mobile phone 7400 is bent by the external force, the secondary battery 7407 provided therein is also bent. FIG. 21C shows the bent secondary battery 7407. The secondary battery 7407 is a thin storage battery. The secondary battery 7407 is fixed in a state of being bent. Note that the secondary battery 7407 includes a lead electrode 7408 electrically connected to a current collector 7409.

FIG. 21D shows an example of a bangle-type display device. A portable display device 7100 includes a housing 7101, a display portion 7102, operation buttons 7103, and a secondary battery 7104. FIG. 21E shows the bent secondary battery 7104. When the display device is worn on a user's arm while the secondary battery 7104 is bent, the housing changes its shape and the curvature of part or the whole of the secondary battery 7104 is changed. Note that a value represented by the radius of a circle that corresponds to the bending condition of a curve at a given point is the radius of curvature, and the reciprocal of the radius of curvature is referred to as curvature. Specifically, part or the whole of the housing or the main surface of the secondary battery 7104 is changed in the range of radius of curvature from 40 mm or more to 150 mm or less. When the radius of curvature at the main surface of the secondary battery 7104 is in the range from 40 mm or more to 150 mm or less, the reliability can be kept high.

FIG. 21F shows an example of a watch-type portable information terminal. A portable information terminal 7200 includes a housing 7201, a display portion 7202, a band 7203, a buckle 7204, an operation button 7205, an input/output terminal 7206, and the like.

The portable information terminal 7200 is capable of executing a variety of applications such as mobile phone calls, e-mailing, viewing and editing texts, music reproduction, Internet communication, and a computer game.

The display surface of the display portion 7202 is curved, and images can be displayed on the curved display surface. In addition, the display portion 7202 includes a touch sensor, and operation can be performed by touching the screen with a finger, a stylus, or the like. For example, by touching an icon 7207 displayed on the display portion 7202, application can be started.

With the operation button 7205, a variety of functions such as time setting, power on/off, on/off of wireless communication, setting and cancellation of a silent mode, and setting and cancellation of a power saving mode can be performed. For example, the functions of the operation button 7205 can be set freely by setting the operation system incorporated in the portable information terminal 7200.

The portable information terminal 7200 can employ near field communication that is a communication method based on an existing communication standard. In that case, for example, mutual communication between the portable information terminal 7200 and a headset capable of wireless communication can be performed, and thus hands-free calling is possible.

Moreover, the portable information terminal 7200 includes the input/output terminal 7206, and data can be directly transmitted to and received from another information terminal through a connector. In addition, charging with the input/output terminal 7206 is possible. Note that the charging operation may be performed by wireless power feeding without using the input/output terminal 7206.

The display portion 7202 of the portable information terminal 7200 includes the secondary battery of one embodiment of the present invention. For example, the secondary battery 7104 shown in FIG. 21E can be provided in the housing 7201 while being curved, or can be provided in the band 7203 such that it can be curved.

The portable information terminal 7200 preferably includes a sensor. As the sensor, for example, a human body sensor such as a fingerprint sensor, a pulse sensor, or a body temperature sensor, a touch sensor, a pressure sensitive sensor, or an acceleration sensor is preferably mounted.

FIG. 21G shows an example of an armband display device. A display device 7300 includes a display portion 7304 and the secondary battery of one embodiment of the present invention. The display device 7300 can include a touch sensor in the display portion 7304 and can serve as a portable information terminal.

The display surface of the display portion 7304 is curved, and images can be displayed on the curved display surface. A display state of the display device 7300 can be changed by, for example, near field communication, which is a communication method based on an existing communication standard.

The display device 7300 includes an input/output terminal, and data can be directly transmitted to and received from another information terminal with a connector. In addition, charging with the input/output terminal is possible. Note that the charge operation may be performed by wireless power feeding without using the input/output terminal.

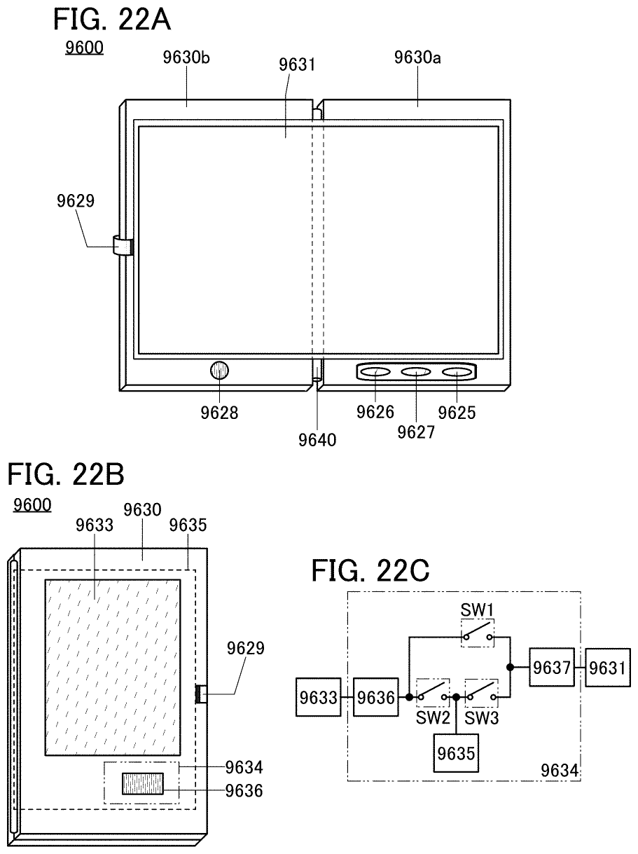

Next, FIG. 22A and FIG. 22B show an example of a tablet terminal that can be folded in half A tablet terminal 9600 shown in FIG. 22A and FIG. 22B includes a housing 9630a, a housing 9630b, a movable portion 9640 connecting the housing 9630a and the housing 9630b, a display portion 9631, a display mode changing switch 9626, a power switch 9627, a power saving mode changing switch 9625, a fastener 9629, and an operation switch 9628. A flexible panel is used for the display portion 9631, whereby a tablet terminal with a larger display portion can be provided. FIG. 22A shows the tablet terminal 9600 that is opened, and FIG. 22B shows the tablet terminal 9600 that is closed.

The tablet terminal 9600 includes a power storage unit 9635 inside the housing 9630a and the housing 9630b. The power storage unit 9635 is provided across the housing 9630a and the housing 9630b over the movable portion 9640.

Part of the display portion 9631 can be a touch panel region and data can be input when a displayed operation key is touched. When a position where a keyboard display switching button is displayed on the touch panel is touched with a finger, a stylus, or the like, keyboard buttons can be displayed on the display portion 9631.

The display mode changing switch 9626 can switch the display between a portrait mode and a landscape mode, and between monochrome display and color display, for example. With the power saving mode changing switch 9625, display luminance can be optimized in accordance with the amount of external light in use, which is detected with an optical sensor incorporated in the tablet terminal 9600. Another detection device including a sensor for detecting inclination, such as a gyroscope sensor or an acceleration sensor, may be incorporated in the tablet terminal, in addition to the optical sensor.

The tablet terminal is closed in FIG. 22B. The tablet terminal includes the housing 9630, a solar cell 9633, and a charging-discharging control circuit 9634 including a DC-DC converter 9636. The secondary battery of one embodiment of the present invention is used as the power storage unit 9635.

Note that the tablet terminal 9600 can be folded in half, thus, the tablet terminal 9600 can be folded so that the housing 9630a and the housing 9630b overlap with each other when not in use. Thus, the display portion 9631 can be protected owing to the holding, which increases the durability of the tablet terminal 9600. With the power storage unit 9635 including the secondary battery of one embodiment of the present invention which has high capacity and excellent cycle characteristics, the tablet terminal which can be used for a long time for a long period can be provided.

The tablet terminal shown in FIG. 22A and FIG. 22B can also have a function of displaying various kinds of information (a still image, a moving image, a text image, and the like), a function of displaying a calendar, a date, the time, or the like on the display portion, a touch-input function of operating or editing data displayed on the display portion by touch input, a function of controlling processing by various kinds of software (programs), and the like.

The solar cell 9633, which is attached on the surface of the tablet terminal, supplies electric power to a touch panel, a display portion, an image signal processor, and the like. Note that the solar cell 9633 can be provided on one surface or both surfaces of the housing 9630 and the power storage unit 9635 can be charged efficiently.

The structure and operation of the charging-discharging control circuit 9634 shown in FIG. 22B are described with reference to a block diagram in FIG. 22C. The solar cell 9633, the power storage unit 9635, the DC-DC converter 9636, a converter 9637, switches SW1 to SW3, and the display portion 9631 are shown in FIG. 22C, and the power storage unit 9635, the DC-DC converter 9636, the converter 9637, and the switches SW1 to SW3 correspond to the charging-discharging control circuit 9634 shown in FIG. 22B.

First, an operation example in which electric power is generated by the solar cell 9633 using external light is described. The voltage of electric power generated by the solar cell is raised or lowered by the DC-DC converter 9636 to a voltage for charging the power storage unit 9635. When the display portion 9631 is operated with the electric power from the solar cell 9633, the switch SW1 is turned on and the voltage is raised or lowered by the converter 9637 to a voltage needed for the display portion 9631. When display on the display portion 9631 is not performed, the switch SW1 is turned off and the switch SW2 is turned on, so that the power storage unit 9635 is charged.

Note that the solar cell 9633 is described as an example of a power generation unit; however, one embodiment of the present invention is not limited to this example. The power storage unit 9635 may be charged using another power generation unit such as a piezoelectric element or a thermoelectric conversion element (Peltier element). For example, the charging may be performed with a non-contact power transmission module that performs charging by transmitting and receiving power wirelessly (without contact), or with a combination of other charging units.

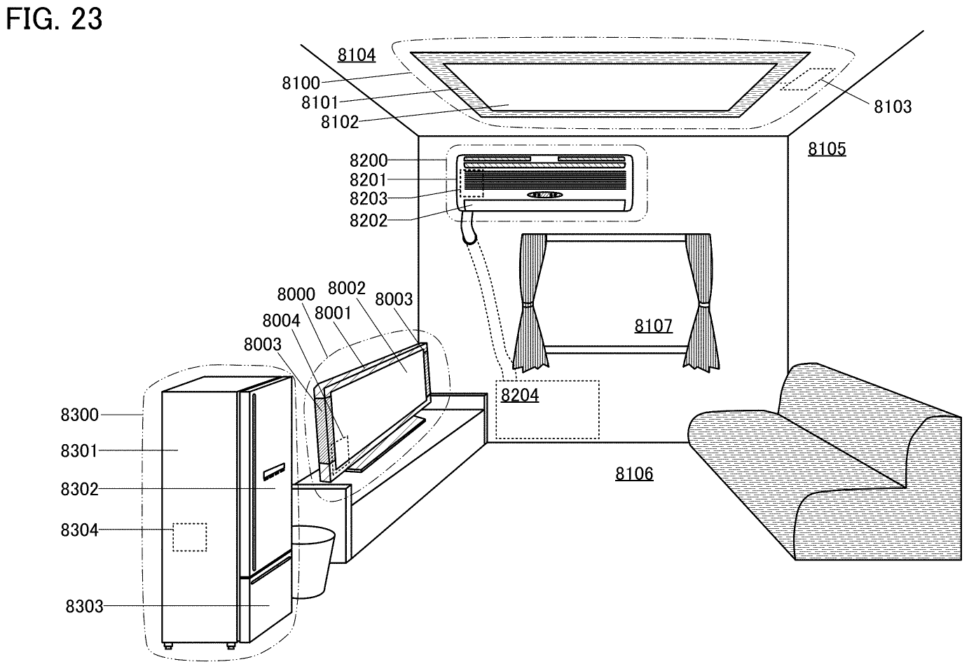

FIG. 23 shows other examples of electronic devices. In FIG. 23, a display device 8000 is an example of an electronic device including a secondary battery 8004 of one embodiment of the present invention. Specifically, the display device 8000 corresponds to a display device for TV broadcast reception and includes a housing 8001, a display portion 8002, speaker portions 8003, the secondary battery 8004, and the like. The secondary battery 8004 of one embodiment of the present invention is provided in the housing 8001. The display device 8000 can receive electric power from a commercial power supply and can use electric power stored in the secondary battery 8004. Thus, the display device 8000 can be operated with the use of the secondary battery 8004 of one embodiment of the present invention as an uninterruptible power supply even when electric power cannot be supplied from a commercial power supply due to power failure or the like.

A semiconductor display device such as a liquid crystal display device, a light-emitting device in which a light-emitting element such as an organic EL element is provided in each pixel, an electrophoresis display device, a DMD (Digital Micromirror Device), a PDP (Plasma Display Panel), or an FED (Field Emission Display) can be used for the display portion 8002.

Note that the display device includes, in its category, all of information display devices for personal computers, advertisement displays, and the like besides TV broadcast reception.

In FIG. 23, an installation lighting device 8100 is an example of an electronic device including a secondary battery 8103 of one embodiment of the present invention. Specifically, the lighting device 8100 includes a housing 8101, a light source 8102, the secondary battery 8103, and the like. Although FIG. 23 shows the case where the secondary battery 8103 is provided in a ceiling 8104 on which the housing 8101 and the light source 8102 are installed, the secondary battery 8103 may be provided in the housing 8101. The lighting device 8100 can receive electric power from a commercial power supply and can use electric power stored in the secondary battery 8103. Thus, the lighting device 8100 can be operated with the use of the secondary battery 8103 of one embodiment of the present invention as an uninterruptible power supply even when electric power cannot be supplied from a commercial power supply due to power failure or the like.

Note that although the installation lighting device 8100 provided in the ceiling 8104 is shown in FIG. 23 as an example, the secondary battery of one embodiment of the present invention can be used in an installation lighting device provided in, for example, a side wall 8105, a floor 8106, or a window 8107 other than the ceiling 8104, and can be used in a tabletop lighting device or the like.

As the light source 8102, an artificial light source that emits light artificially by using electric power can be used. Specifically, an incandescent lamp, a discharge lamp such as a fluorescent lamp, and light-emitting elements such as an LED and an organic EL element are given as examples of the artificial light source.

In FIG. 23, an air conditioner including an indoor unit 8200 and an outdoor unit 8204 is an example of an electronic device including a secondary battery 8203 of one embodiment of the present invention. Specifically, the indoor unit 8200 includes a housing 8201, an air outlet 8202, the secondary battery 8203, and the like. Although FIG. 23 shows the case where the secondary battery 8203 is provided in the indoor unit 8200, the secondary battery 8203 may be provided in the outdoor unit 8204. Alternatively, the secondary batteries 8203 may be provided in both the indoor unit 8200 and the outdoor unit 8204. The air conditioner can receive electric power from a commercial power supply and can use electric power stored in the secondary battery 8203. Particularly in the case where the secondary batteries 8203 are provided in both the indoor unit 8200 and the outdoor unit 8204, the air conditioner can be operated with the use of the secondary battery 8203 of one embodiment of the present invention as an uninterruptible power supply even when electric power cannot be supplied from a commercial power supply due to power failure or the like.

Note that although the split-type air conditioner including the indoor unit and the outdoor unit is illustrated in FIG. 23 as an example, the secondary battery of one embodiment of the present invention can be used in an air conditioner in which the function of an indoor unit and the function of an outdoor unit are integrated in one housing.

In FIG. 23, an electric refrigerator-freezer 8300 is an example of an electronic device including a secondary battery 8304 of one embodiment of the present invention. Specifically, the electric refrigerator-freezer 8300 includes a housing 8301, a refrigerator door 8302, a freezer door 8303, the secondary battery 8304, and the like. The secondary battery 8304 is provided in the housing 8301 in FIG. 23. The electric refrigerator-freezer 8300 can receive electric power from a commercial power supply and can use electric power stored in the secondary battery 8304. Thus, the electric refrigerator-freezer 8300 can be operated with the use of the secondary battery 8304 of one embodiment of the present invention as an uninterruptible power supply even when electric power cannot be supplied from a commercial power supply due to power failure or the like.

In addition, in a time period when electronic devices are not used, particularly when the proportion of the amount of electric power which is actually used to the total amount of electric power which can be supplied from a commercial power supply source (such a proportion is referred to as a usage rate of electric power) is low, electric power is stored in the secondary battery, whereby the usage rate of electric power can be reduced in a time period other than the above time period. For example, in the case of the electric refrigerator-freezer 8300, electric power is stored in the secondary battery 8304 in night time when the temperature is low and the refrigerator door 8302 and the freezer door 8303 are not opened or closed. On the other hand, in daytime when the temperature is high and the refrigerator door 8302 and the freezer door 8303 are opened and closed, the secondary battery 8304 is used as an auxiliary power supply; thus, the usage rate of electric power in daytime can be reduced.

The secondary battery of one embodiment of the present invention can be used in a variety of electronic devices as well as the above electronic devices. According to one embodiment of the present invention, the secondary battery can have little deterioration and high safety. Thus, when the secondary battery of one embodiment of the present invention is used in the electronic devices described in this embodiment, electronic devices with longer lifetime and higher safety can be obtained. This embodiment can be implemented in appropriate combination with the other embodiments.

### Embodiment 6

In this embodiment, examples of vehicles each including the secondary battery of one embodiment of the present invention are described.

The use of secondary batteries in vehicles enables production of next-generation clean energy vehicles such as hybrid electric vehicles (HEVs), electric vehicles (EVs), and plug-in hybrid electric vehicles (PHEVs).

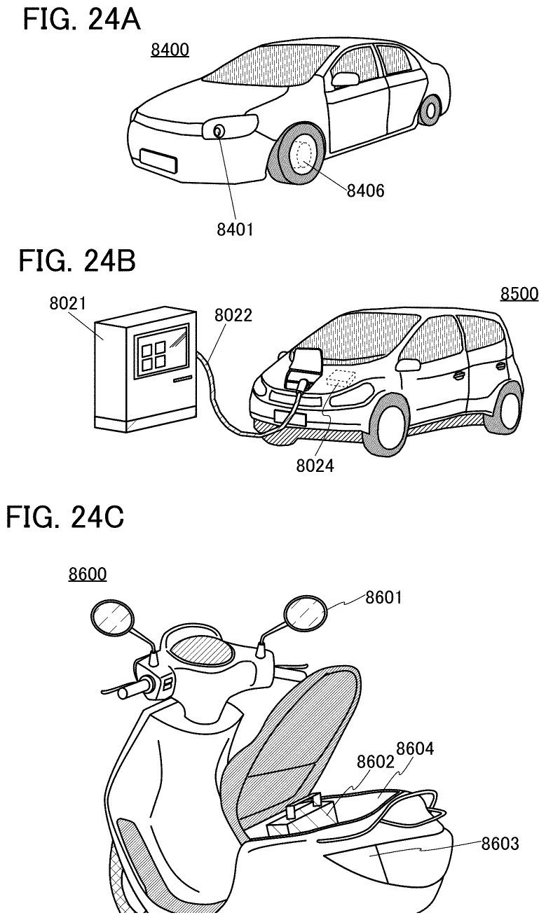

FIG. 24 shows examples of vehicles each using the secondary battery of one embodiment of the present invention. An automobile 8400 shown in FIG. 24A is an electric vehicle that runs on an electric motor as a power source. Alternatively, the automobile 8400 is a hybrid electric vehicle capable of running on the power of either an electric motor or an engine as appropriate. The use of one embodiment of the present invention allows fabrication of a high-mileage vehicle. The automobile 8400 includes a secondary battery. The secondary battery is used not only for driving an electric motor 8406, but also for supplying electric power to a light-emitting device such as a headlight 8401 or a room light (not illustrated).

The secondary battery can also supply electric power to a display device included in the automobile 8400, such as a speedometer or a tachometer. Furthermore, the secondary battery can supply electric power to a semiconductor device included in the automobile 8400, such as a navigation system.

FIG. 24B shows an automobile 8500 including the secondary battery. The automobile 8500 can be charged when the secondary battery is supplied with electric power with external charging equipment of a plug-in system, a contactless power feeding system, or the like. In FIG. 24B, a secondary battery 8024 included in the automobile 8500 are charged with the use of a ground-based charging apparatus 8021 with a cable 8022. In charging, a given method such as CHAdeMO (registered trademark) or Combined Charging System may be employed as a charging method, the standard of a connector, or the like as appropriate. The charging apparatus 8021 may be a charging station provided in a commerce facility or a power source in a house. For example, with the use of a plug-in technique, the secondary battery 8024 included in the automobile 8500 can be charged by being supplied with electric power from outside. The charging can be performed by converting AC electric power into DC electric power with a converter such as an AC-DC converter.

Furthermore, although not shown, the vehicle may include a power receiving device so that it can be charged by being supplied with electric power from an above-ground power transmitting device in a contactless manner. In the case of the contactless power feeding system, by fitting a power transmitting device in a road or an exterior wall, charging can be performed not only when the vehicle is stopped but also when driven. In addition, the contactless charging system may be utilized to perform transmission and reception of electric power between vehicles. Furthermore, a solar cell may be provided in the exterior of the vehicle to charge the secondary battery when the vehicle stops or moves. To supply electric power in such a contactless manner, an electromagnetic induction method or a magnetic resonance method can be used.

FIG. 24C shows an example of a motorcycle including the secondary battery of one embodiment of the present invention. A motor scooter 8600 shown in FIG. 24C includes a secondary battery 8602, side mirrors 8601, and indicators 8603. The secondary battery 8602 can supply electric power to the direction indicators 8603.

Furthermore, in the motor scooter 8600 shown in FIG. 24C, the secondary battery 8602 can be held in a storage unit under seat storage 8604. The secondary battery 8602 can be held in the storage unit under seat storage 8604 even when the storage unit under seat storage 8604 is small.

According to one embodiment of the present invention, the secondary battery can have little deterioration and high safety. Thus, when the secondary battery is mounted on a vehicle, a reduction in mileage, acceleration performance, or the like can be inhibited. In addition, a highly safe vehicle can be achieved. Furthermore, the secondary battery included in the vehicle can be used as a power source for supplying electric power to products other than the vehicle. In such a case, the use of a commercial power supply can be avoided at peak time of electric power demand, for example. Avoiding the use of a commercial power supply at peak time of electric power demand can contribute to energy saving and reduction in carbon dioxide emissions. Moreover, the secondary battery with little deterioration and high safety can be used for a long period; thus, the use amount of rare metals such as cobalt can be reduced.

This embodiment can be implemented in appropriate combination with the other embodiments.

### Example 1

In this example, an analysis of substitution positions of a Ni atom and a Mg atom in a compound represented by the chemical formula Li_{(1-x-y)}Co_{(1-a-b)}Ni₍ₓ₊ₐ₎Mg_{(y+b)}O₂ is described.

### [Calculation of Stabilization Energy (ΔE) Different Between Substitution Positions]

Li_{(1-x-y)}Co_{(1-a-b)}Ni₍ₓ₊ₐ₎Mg_{(y+b)}O₂ is a compound in which Mg and Ni are added to LiCoO₂ as substitution elements. When the one Mg atom and the one Ni atom are substituted for metal atoms contained in LiCoO₂, combinations of substitution sites are presumably the above combinations. These are described again below.

Li in the same Li layer are substituted by Mg and Ni: Condition (A).
Li in different Li layers are substituted by Mg and Ni: Condition (B).
Co in the same Co layer are substituted by Mg and Ni: Condition (C).
Co in different Co layers are substituted by Mg and Ni: Condition (D).
Li in a Li layer is substituted by Mg and Co in a Co layer is substituted by Ni: Condition (E).
Li in a Li layer is substituted by Ni and Co in a Co layer is substituted by Mg: Condition (F).

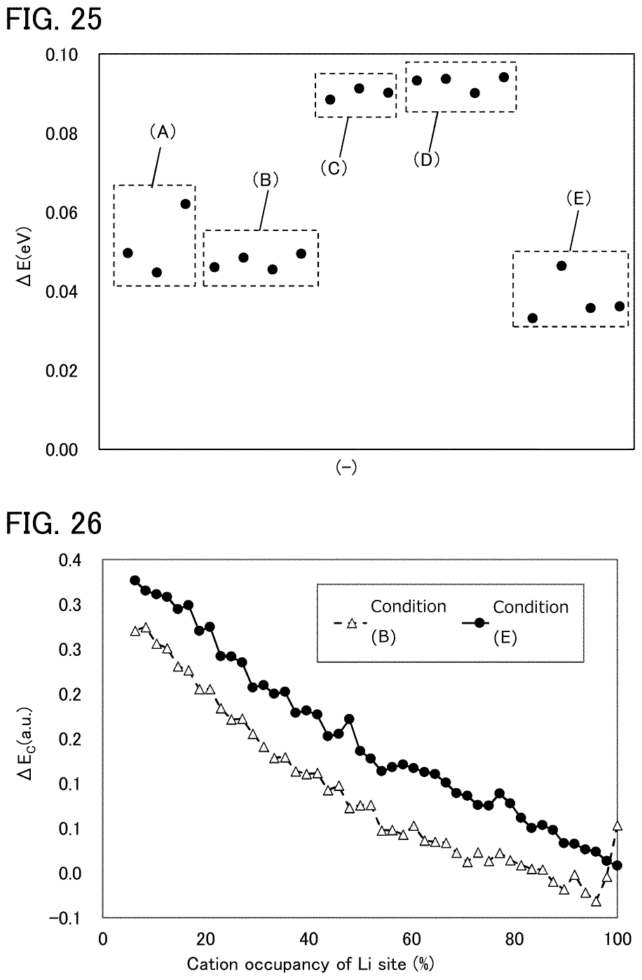

As for the combinations of the substitution sites above, the stabilization energy ΔE of the combinations in which Ni and Mg have a relation of the first proximity to the third or the fourth proximity was calculated using Formulae 1 to 3. The calculation conditions of LiCoO₂ are shown in Table 1 and the calculation results of ΔE are shown in FIG. 25. Note that (A) to (E) in FIG. 25 show the calculation results of Conditions (A) to (E) above. A Mg ion has the larger ion radius than a Co ion; it is expected that Mg needs large energy to substitute a Co site. Thus, Condition (F) presumably has large stabilization energy.

Note that VSAP in Table 1 shows Vienna Ab initio Simulation Package (bought from VASP Software GmbH).

TABLE 1

| Software | VASP | |
|---|---|---|
| Functional | GGA + U(DFT-D2) | |
| Pseudo-potential | PAW | |
| Cut-off energy (eV) | 600 | |
| U potential | Co | 4.91 |

| | |
|---|---|
| Number of atoms | Li: 48, Co: 48, O: 96 |
| k-points | 1 × 1 × 1 |
| Calculation target | Lattice and atom position are optimized |

FIG. 25 showed that the stabilization energy of Conditions (A), (B), and (E) tended to be small and Conditions (C) and (D) had large stabilization energy and were unstable conditions. It was suggested that Mg and Ni were not easily substituted for Co sites. It was also suggested that Condition (E) had the smallest stabilization energy and Ni in a Co site was stabilized by Mg substituted for a Li site. It was also suggested that Conditions (A) and (B) had stabilization energy in a similar level, which were slightly larger than that of Condition (E).

[Calculation of Stabilization Energy (ΔE_{C}) when Cation Occupancy of Li Sites is Changed]

ΔE_{C} was calculated to estimate energy change in charging. According to FIG. 25, the crystal structure of a compound represented by the chemical formula Li_{(1-x-y)}Co_{(1-a-b)}Ni₍ₓ₊ₐ₎Mg_{(y+b)}O₂ probably corresponds to Conditions (A), (B), and (E). Each ΔE_{C} of Conditions (B) and (E) was calculated using Formulae (4) and (5). FIG. 26 shows a plot of ΔE_{C} with respect to cation occupancy of Li sites. Using FIG. 25, calculation cost was reduced.

According to FIG. 26, Condition (E) shows the relation in which as the cation occupancy of Li sites decreases, ΔE_{C} constantly increases. It was shown that when an occupancy of Li sites was 100%, which was a state before charging, ΔE_{C} of Condition (E) was smaller than that of Condition (B); when an occupancy of Li sites was 98% or less, ΔE_{C} of Condition (B) was smaller than that of Condition (E). It was also shown that Condition (B) had a local minimum point when an occupancy of Li sites was 96%. This suggested that Condition (B) was more stable after charging started.

The above results suggest that in a compound represented by the chemical formula Li_{(1-x-y)}Co_{(1-a-b)}Ni₍ₓ₊ₐ₎Mg_{(y+b)}O₂, Mg and Ni are substituted for a Li site and a Co site respectively immediately after the synthesis, and Ni moves to a Li site to relax instability through charging. That is, it is suggested that the substitution position of Ni changed between before and after charging. In other words, it is suggested that the event of a Ni movement from a Co site to a Li site by charging occurred.

### [Measurement of Charge-Discharge Efficiency]

To confirm whether the event of a Ni movement from a Co site to a Li site by charging actually occurs or not, a compound represented by the chemical formula Li_{(1-x-y)}Co_{(1-a-b)}Ni₍ₓ₊ₐ₎Mg_{(y+b)}O₂ was made and charge-discharge efficiency of a secondary battery using the compound was measured.

<Making of a Compound Represented by Chemical Formula Li_{(1-x-y)}Co_{(1-a-b)}Ni₍ₓ₊ₐ₎Mg_{(y+b)}O₂>

A compound represented by the chemical formula Li_{(1-x-y)}Co_{(1-a-b)}Ni₍ₓ₊ₐ₎Mg_{(y+b)}O₂ was made as the positive electrode active material 100A-1 with reference to the flowchart in FIG. 3. Sample 1 to Sample 3, which differed in the combining amount of nickel hydroxide Ni(OH)2, were made. Two similar samples for each Sample were made and charge-discharge efficiency of Sample 1 to Sample 3 was measured twice.

First, the mixture 902 containing magnesium and fluorine was formed (Step S11 to Step S14). First, LiF and MgF₂ were weighted so that the molar ratio of LiF to MgF₂ was LiF:MgF₂=1:3, acetone was added as a solvent, and the materials were mixed and ground by a wet process. The mixing and the grinding were performed in a ball mill using a zirconia ball at 400 rpm for 12 hour. The material that has been subjected to the treatment was collected to be the mixture 902.

Next, nickel hydroxide, which is a metal source, and acetone were mixed to form pulverized nickel hydroxide (Step S15 to Step S17).

Next, lithium cobaltate was prepared as a composite oxide containing lithium and cobalt. Specifically, CELLSEED C-10N manufactured by NIPPON CHEMICAL INDUSTRIAL CO., LTD. was prepared (Step S25).

Next, in Step S31, the materials were weighed so that the number of atoms of magnesium in the mixture 902 was 2.0 mol % of the number of atoms of cobalt in the lithium cobaltate. The mixing was performed by a dry method. The mixing was performed in a ball mill using a zirconia ball at 150 rpm for 1 hour.

Next, each of the mixtures 903 was put in an alumina crucible and annealed at 850° C. using a muffle furnace in an oxygen atmosphere for 60 hours (Step S34). At the time of annealing, the alumina crucible was covered with a lid. The flow rate of oxygen was 10 L/min. The temperature rise was 200° C./hr, and it took longer than or equal to 10 hours to lower the temperature. The material subjected to the heat treatment was collected and filtered (Step S35), and the mixture 904 was obtained (Step S36).

Next, nickel hydroxide, which is a metal source, and acetone were mixed to form pulverized nickel hydroxide (Step S15 to Step S17).

Next, in Step S50, nickel hydroxide and the mixture 903 were weighed so that the number of nickel atoms contained in nickel hydroxide was w mol % of the sum of the number of cobalt atoms and the number of nickel atoms contained in the mixture 903. Each sample has different w, as shown in Table 2 below. The weighed mixture 903 and nickel hydroxide were mixed. The mixing was performed by a dry method. The mixing was performed in a ball mill using a zirconia ball at 150 rpm for 1 hour.

TABLE 2

| Sample name | w (mol %) |
|---|---|
| Sample 1 | 0.1 |
| Sample 2 | 0.5 |
| Sample 3 | 2.0 |

Next, the materials that has been subjected to the treatment were collected to obtain the mixtures 905 (Step S51 and Step S52).

Next, the mixture 905 was put in an alumina crucible and annealed at 850° C. using a muffle furnace in an oxygen atmosphere for 60 hours (Step S53). At the time of annealing, the alumina crucible was covered with a lid. The flow rate of oxygen was 10 L/min. The temperature rise was 200° C./hr, and it took longer than or equal to 10 hours to lower the temperature. The material after the heat treatment was collected and filtered (Step S54), and Sample 1 to Sample 3 were obtained (Step S55).

### <Making of Battery Cell>

Next, Sample 1 to Sample 3 obtained above were used as positive electrode active materials to make respective positive electrodes. A current collector that was coated with slurry in which the positive electrode active material, AB, and PVDF were mixed at the active material:AB:PVDF=95:3:2 (weight ratio) was used. As a solvent of the slurry, NMP was used.

After the current collector was coated with the slurry, the solvent was volatilized. Then, pressure was applied at 210 kN/m, and then pressure was applied at 1467 kN/m. Through the above process, the positive electrode was obtained. The load amount of the positive electrode was approximately 7 mg/cm² and an electrode density was 3.8 g/cc.

Using the fabricated positive electrodes, CR2032 type coin battery cells (diameter: 20 mm, height: 3.2 mm) were fabricated.

A lithium metal was used for a counter electrode.

As an electrolyte contained in the electrolyte solution, 1 mol/L lithium hexafluorophosphate (LiPF₆) was used. As the electrolyte solution, an electrolyte solution in which ethylene carbonate (EC) and diethyl carbonate (DEC) were mixed at EC:DEC=3:7 (volume ratio) was used. Note that for secondary batteries used for evaluating the charge-discharge efficiency, 2 wt % of vinylene carbonate (VC) was added to the electrolytic solution.

As a separator, 25-μm-thick polypropylene was used.

A positive electrode can and a negative electrode can that are formed using stainless steel (SUS) were used.

### <Measurement of Charge-Discharge Efficiency>

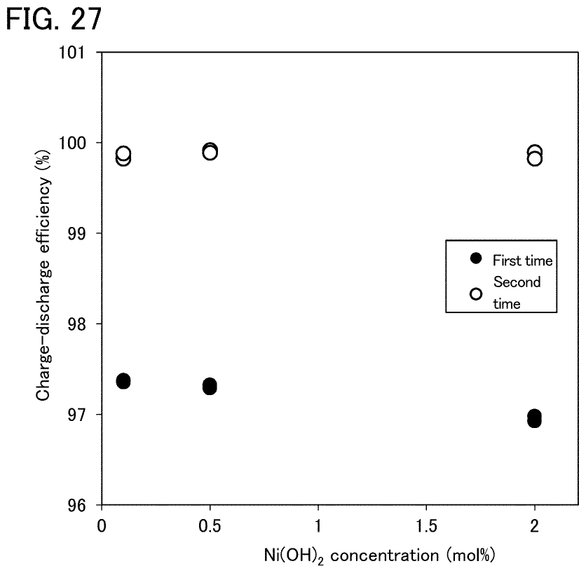

The charge-discharge efficiency in the first cycle and that in the second cycle of the battery cells using obtained Sample 1 to Sample 3 were measured. FIG. 27 shows the result. As measurement conditions of the charge-discharge efficiency, CCCV charging (0.5 C, 4.6 V, a termination current of 0.05 C) and CC discharging (0.5 C, 2.5 V) were repeatedly performed at 25° C. C rate was set to approximately 200 μmA/g. Note that the following is satisfied: charge-discharge efficiency (%)=(discharge capacity/charge capacity)×100.

FIG. 27 shows that Sample 1 to Sample 3 had the charge-discharge efficiency in the first cycle of less than 100%. This result suggests that Ni substituted for a Co layer before charging moves to another site by charging. Analysis was conducted with the calculation result, and the event in which Ni moved from a Co site to a Li site by charging presumably occurred. The charge-discharge efficiency in the first cycle tends to decrease as the addition amount of Ni(OH)₂ increases. The discharge capacity is presumably decreased by Ni movement to a Li site; a sample with a higher Ni concentration is expected to have a larger decrease (difference from 100%) in charge-discharge efficiency. That is, the tendency presumably reflects the event. The charge-discharge efficiency in the second cycle of Sample 1 to Sample 3 showed approximately 100%. Thus, it was suggested that the event was irreversible and occurs at the first charging.

The calculation result and the measurement result suggest that, according to only FIG. 25, a compound represented by the chemical formula Li_{(1-x-y)}Co_{(1-a-b)}Ni₍ₓ₊ₐ₎Mg_{(y+b)}O₂ is the stablest when the compound has the structure in which Mg is substituted for a Li site and Ni is substituted for a Co site; however, according to the ΔE_{C} calculations and the measurement results of charge-discharge efficiency, the compound after charging is stabler when Mg and Ni are substituted for Li sites. Thus, it was suggested that the compound had a structure in which Mg and Ni were substituted for a Li site and a Co site respectively at the synthesis, and by charging, Ni moved to a Li site and the structure changed. By not only calculating ΔE but also calculating ΔE_{C} and measuring charge-discharge efficiency, or not only calculation but also actual measurement as described above, the structure of a lithium composite oxide can be analyzed more accurately with both calculation and experiment. It was found that ΔE_{C} was efficiently calculated through calculation of ΔE, which reduced calculation cost. With calculation, the validity of the events occurring in the compound can be evaluated, which enables an efficient measurement. Thus, the number of samples and time for measurement can be reduced.

### REFERENCE NUMERALS

100: positive electrode active material, 100A-1: positive electrode active material, 100C: positive electrode active material, 200: active material layer, 201: graphene compound, 211a: positive electrode, 211b: negative electrode, 212a: lead, 212b: lead, 214: separator, 215a: bonding portion, 215b: bonding portion, 217: fixing member, 250: battery, 251: exterior body, 261: folded portion, 262: seal portion, 263: seal portion, 271: crest line, 272: crest line, 273: space, 300: secondary battery, 301: positive electrode can, 302: negative electrode can, 303: gasket, 304: positive electrode, 305: positive electrode current collector, 306: positive electrode active material layer, 307: negative electrode, 308: negative electrode current collector, 309: negative electrode active material layer, 310: separator, 500: secondary battery, 501: positive electrode current collector, 502: positive electrode active material layer, 503: positive electrode, 504: negative electrode current collector, 505: negative electrode active material layer, 506: negative electrode, 507: separator, 508: electrolyte, 509: exterior body, 510: positive electrode lead electrode, 511: negative electrode lead electrode, 600: secondary battery, 601: positive electrode cap, 602: battery can, 603: positive electrode terminal, 604: positive electrode, 605: separator, 606: negative electrode, 607: negative electrode terminal, 608: insulating plate, 609: insulating plate, 611: PTC element, 612: safety valve mechanism, 613: conductive plate, 614: conductive plate, 615: module, 616: wiring, 617: temperature control device, 900: circuit substrate, 902: mixture, 903: mixture, 904: mixture, 905: mixture, 910: label, 911: terminal, 912: circuit, 913: secondary battery, 914: antenna, 915: antenna, 916: layer, 917: layer, 918: antenna, 919: terminal, 920: display device, 921: sensor, 922: terminal, 930: housing, 930a: housing, 930b: housing, 931: negative electrode, 932: positive electrode, 933: separator, 950: wound body, 951: terminal, 952: terminal, 980: secondary battery, 981: film, 982: film, 993: wound body, 994: negative electrode, 995: positive electrode, 996: separator, 997: lead electrode, 998: lead electrode, 7100: portable display device, 7101: housing, 7102: display portion, 7103: operation button, 7104: secondary battery, 7200: portable information terminal, 7201: housing, 7202: display portion, 7203: band, 7204: buckle, 7205: operation button, 7206: input/output terminal, 7207: icon, 7300: display device, 7304: display portion, 7400: mobile phone, 7401: housing, 7402: display portion, 7403: operation button, 7404: external connection port, 7405: speaker, 7406: microphone, 7407: secondary battery, 7408: lead electrode, 7409: current collector, 8000: display device, 8001: housing, 8002: display portion, 8003: speaker portion, 8004: secondary battery, 8021: charging device, 8022: cable, 8024: secondary battery, 8100: lighting device, 8101: housing, 8102: light source, 8103: secondary battery, 8104: ceiling, 8105: side wall, 8106: floor, 8107: window, 8200: indoor unit, 8201: housing, 8202: air outlet, 8203: secondary battery, 8204: outdoor unit, 8300: electric refrigerator-freezer, 8301: housing, 8302: refrigerator door, 8303: freezer door, 8304: secondary battery, 8400: automobile, 8401: headlight, 8406: electric motor, 8500: automobile, 8600: scooter, 8601: side mirror, 8602: secondary battery, 8603: direction indicator, 8604: storage unit under seat storage, 9600: tablet terminal, 9625: switch, 9626: display mode changing switch, 9627: power switch, 9628: operation switch, 9629: fastener, 9630: housing, 9630a: housing, 9630b: housing, 9631: display portion, 9633: solar cell, 9634: charging-discharging control circuit, 9635: power storage unit, 9636: DC-DC converter, 9637: converter, 9640: movable portion

1. A method to analyze substitution positions of a Ni atom and a Mg atom in a compound represented by a chemical formula Li_{(1-x-y)}Co_{(1-a-b)}Ni₍ₓ₊ₐ₎Mg_{(y+b)}O₂ comprising:
a first calculation step of calculating stabilization energy of the compound represented by the chemical formula when the Ni atom and the Mg atom each independently substitute for Li atoms contained in a LiCoO2 crystal, the Ni atom and the Mg atom each independently substitute for Co atoms contained in a LiCoO₂ crystal, and the Ni atom and the Mg atom each independently substitute for a Li atom and a Co atom contained in a LiCoO₂ crystal;
a second calculation step of calculating the stabilization energy of the compound represented by the chemical formula when cation occupancy of Li sites is changed; and
a first measurement step of measuring charge-discharge efficiency in the first cycle and charge-discharge efficiency in the n-th cycle of the compound represented by the chemical formula,
wherein, in the chemical formula, x+y<1, a+b<1, and x, y, a, and b each independently represent a real number greater than or equal to 0 and less than or equal to 1, and,
wherein n is an integer greater than or equal to 2.

2. The method to analyze substitution positions of a Ni atom and a Mg atom according to claim 1,
wherein in the first calculation step and the second calculation step, a GGA+U(DFT-D2) method is used.

3. The method to analyze substitution positions of a Ni atom and a Mg atom according to claim 1,
wherein the cation occupancy is changed at least within a range of 80% to 100% for calculation.

4. The method to analyze substitution positions of a Ni atom and a Mg atom according to claim 1,
wherein n=2.

5. The method to analyze substitution positions of a Ni atom and a Mg atom according to claim 1,
wherein in the chemical formula, 0<x+a≤0.015 and 0<y+b≤0.06.

6. The method to analyze substitution positions of a Ni atom and a Mg atom according to claim 1, further comprising a step in which, in the second calculation step, when the Ni atom and the Mg atom substitute for the Li atoms in the LiCoO₂ crystal and the Ni atom and the Mg atom substitute for the Co atoms in the LiCoO₂ crystal, the stabilization energy of the case where the Li atoms or the Co atoms that exist in the same layer in the LiCoO₂ crystal are substituted by the Ni atom and the Mg atom and the stabilization energy of the case where the Li atoms or the Co atoms that exist in different layers in the LiCoO₂ crystal are substituted by the Ni atom and the Mg atom are calculated.
